(19) United States
(12) Patent Application Publication
(10) Pub. No.: US 2024/0390285 A1
(43) Pub. Date: Nov. 28, 2024

(54) HIGH-DOSE COMPRESSIBLE DOSAGE FORMS MANUFACTURED BY SIMULTANEOUS MELT-COATING AND MELT-GRANULATION OF ACTIVE PHARMACEUTICAL INGREDIENTS

(71) Applicants: Rutgers, The State University of New Jersey, New Brunswick, NJ (US); Janssen Research & Development, LLC, Raritan, NJ (US)

(72) Inventors: Fernando J. Muzzio, Sparta, NJ (US); Ivana M. Cotabarren, Buenos Aires (AR); Shashwat Gupta, North Brunswick, NJ (US); Qiushi Zhou, New Brunswick, NJ (US); Thamer A. Omar, Piscataway, NJ (US); James Scicolone, South Plainfield, NJ (US); Eric J. Sánchez-Rolon, Gurabo, PR (US); Vipul Dave, Hillsborough, NJ (US); George Oze, Washington Station, NJ (US)

(73) Assignees: Rutgers, The State University of New Jersey, New Brunswick, NJ (US); Janssen Research & Development, LLC, Raritan, NJ (US)

(21) Appl. No.: 18/694,042

(22) PCT Filed: Sep. 23, 2022
(86) PCT No.: PCT/US2022/076965

§ 371 Date: Mar. 21, 2024

Related U.S. Application Data
(60) Provisional application No. 63/247,560, filed on Sep. 23, 2021.

(51) Int. Cl. A61K 9/16; A61K 9/20; A61K 31/341; A61K 45/00

(52) U.S. Cl. CPC A61K 9/1641; A61K 9/1694; A61K 9/2018; A61K 9/2027; A61K 9/2054; A61K 31/341; A61K 45/00

(57) ABSTRACT

The present disclosure relates to a process for manufacturing an oral pharmaceutical dosage form including: mixing an active pharmaceutical ingredient (API) and surfactant into a blend; feeding the blend into a processor that applies heat and shear forces at a temperature within a range of approximately equal to the melting point of the surfactant to 3° C. below the melting point of the surfactant so as to form API granulates; and formulating the API granulates into a dosage form. The disclosed technology provides a surprisingly effective and economical means for producing high dose solid dosage forms containing poorly soluble APIs with minimal excipient burden.

### FIELD

The disclosed technology generally relates to the field of pharmaceutical formulation and manufacturing, including pharmaceutical compositions and manufacturing methods for simultaneously melt-coating and melt-granulating active pharmaceutical ingredients (APIs) to produce finished drug products with enhanced properties. More specifically, the disclosed technology provides a surprisingly effective and economical means for producing high dose solid dosage forms from granulates of poorly soluble APIs, wherein such granulates exhibit superior flowability and compressibility, and wherein the resulting dosage forms exhibit superior density, hardness and dissolution with minimized excipient burden.

### BACKGROUND

A large percentage of APIs and new chemical entities have been reported as being poorly water-soluble. In general, poorly water-soluble APIs present challenges such as poor absorption and low bioavailability, which makes it difficult to deliver the API into a subject's blood stream. The US Pharmacopeia describes the solubility of drugs in terms of the number of milliliters of solvent in which one gram of solute will dissolve. Typically, drugs defined as “poorly soluble” are those that require more than 1 ml of solvent per 10 mg of solute. The Biopharmaceutics Classification System (BCS) divides drugs into the following four groups with respect to solubility and permeability: Class I (high solubility, high permeability), Class II (low solubility, high permeability), Class III (high solubility, low permeability), and Class IV (low solubility, low permeability). According to the BCS, a drug substance is considered “poorly soluble” or of low solubility when more than 250 mL of an aqueous solution in a pH range of 1.2 to 6.8 at 37±1° C. is required to solubilize the highest single therapeutic dose. Permeability is evaluated with respect to the extent of absorption of a drug from human pharmacokinetic studies. A drug is considered “highly permeable” when its absolute bioavailability is greater than or equal to 85%. Among the four BCS groups, drugs in Classes II and IV exhibit poor aqueous solubility, resulting in poor bioavailability. Such poorly soluble drugs also often exhibit uneven absorption, with the degree of unevenness being influenced by factors such as dose level, patient satiety, and drug form, as well as a number of other parameters along the manufacturing process affecting the properties of the final drug product.

In the field of pharmaceutical drug development, oral administration is a preferred route for drug delivery due to several advantages such as low cost, pain avoidance, patient convenience, and safety. Thus, there is often a need to improve solubility and bioavailability, including oral bioavailability, of pharmaceutical drug products containing poorly soluble APIs. Prior methods of addressing challenges associated with poorly soluble APIs include micronization, amorphization, spray drying and hot melt extrusion, but such methods tend to have significant disadvantages, such as high cost, difficulty of implementation, instability, and/or dosage limitations. For example, micronization can cause a drug substance to be poorly compressible and poorly flowable, and reduction in particle size can increase the tendency of the drug to undesirably agglomerate. Spray drying and hot melt extrusion processes are complex, expensive to implement and operate, and can result in dosage forms that contain large amounts of excipients, thus limiting the amount of drug that can be loaded into the product units. Accordingly, such techniques are unsuitable for high-dose drug products because the large amount of required excipients results in a dosage form that is too large to be swallowed easily, or that must be administered the patient in multiple dosage forms simultaneously, which increases expense and reduces patient compliance.

Additionally, direct compression is a common and economical technique for manufacturing oral tablets in the pharmaceutical industry. Yet, there are limitations associated with this technique. For example, direct compression may be performed using dry binders or other excipients to enhance the flowability of the component powder and to provide sufficient cohesiveness or compressibility required to form satisfactory tablets of acceptable hardness and density. However, the inclusion of excipients (i.e., a high excipient burden) can limit the total amount of API that may be included in a desirably sized tablet. Hence, high-dose tablets can be difficult to produce by direct compression, particularly if the API itself is not easily compressible.

Thus, there is strong need in the pharmaceutical industry for improved methods of formulating high-dose poorly soluble drugs using simple processes and minimum amounts of excipients. The disclosed technology addresses one or more of the foregoing needs by providing a process for simultaneously melt-coating and melt-granulating poorly soluble APIs to produce API granulates having significantly enhanced flowability, compressibility and compaction with minimized excipient burden, whereby the resulting solid dosage forms produced therefrom are easily administered, contain a high-dose of the API, and exhibit various advantageous properties, such as enhanced dissolution and release profiles and increased density.

### SUMMARY

The present disclosure relates to a surprisingly effective and efficient method of producing high dose solid dosage forms from granulates of poorly soluble APIs by employing a process of simultaneously coating and granulating the drug particles. In some embodiments, the poorly soluble drug is combined, optionally pre-blended, with a small amount (e.g., up to 10 wt %) of a low melting point surfactant, and the combination is then fed into an extruder at a processing temperature that is at or very close to the melting point of the surfactant. As used herein, a “low melting point surfactant” refers to a surfactant having a melting point that is substantially lower than the melting point of the API, e.g., 10° C. lower or more. This process causes the surfactant to soften or partially melt so as to coat the outer surface of at least a portion of the drug particles (see PCT/US21/022533, which is hereby incorporated by reference in its entirety) and to also cause the coated API particles to agglomerate, forming API granulates. The presence of the surfactant on the surface of the drug particles promotes wetting, and greatly enhances dissolution of the poorly soluble drug.

The temperature of the process is critical. While not wishing to be bound by one particular theory, it is believed that by using a process temperature close to the melting point of the surfactant, particularly with a short process time, the combination of heat and mechanical shear causes only a small fraction of the surfactant to soften or melt, which is sufficient in most cases to enable substantial coating of the drug particles, and which promotes agglomeration of such particles into low density granules that dissolve very rapidly compared to the drug being devoid of surfactant. On the other hand, if the process temperature is substantially below the melting point of the surfactant, no coating takes place; and if the process temperature substantially exceeds the melting point of the surfactant, the majority or even the entirety of the surfactant melts, enabling the densification of the resulting granules, which would then dissolve more slowly than desired.

In some embodiments of the disclosed technology, APIs can be melt-coated concurrently with additional low-melting-point substances to control the API release rate, mask unpleasant taste or smell, improve chemical and/or physical stability of the granulate or finished drug product, reduce moisture sensitivity or light sensitivity, and maintain a desired pH, among other applications.

In particular, it has been surprisingly discovered that, when the temperature is maintained close to the melting point of the surfactant, the disclosed process will simultaneously coat and granulate the poorly soluble API, converting the API into rapidly-dissolving, highly compressible low density granulates that can then be optionally mixed with a small amount of controlled release agent—e.g., up to 5 wt % of a sustained release agent such as hydroxypropyl methylcellulose (HPMC), or up to 5 wt % of an immediate release agent (e.g., a disintegrant, such as sodium carboxymethyl cellulose (CMC-Na or NaCMC)—and/or a small amount of lubricant (e.g., up to 0.5 wt % magnesium stearate (MgSt)), and formulated into a finished drug product, such as a compressed tablet. The drug products produced therefrom possess excellent quality attributes with release profiles that can be selected to provide API release ranging from immediate release to sustained release, such as 12-hour release.

The disclosed process is easy to implement and much less expensive than other methods. Further, since the disclosed process does not rely on dispersing the drug in a matrix of another ingredient (as is the case, for example, in spray drying, hot melt extrusion, or other co-processing methods) it is possible to make tablets, capsules and other dosage forms that are remarkably compact and almost entirely composed of the API with only a minimal amount of excipient(s). Consequently, the disclosed process in its various embodiments is suitable for achieving high drug loading of a high density, compact unit dose. In some embodiments, the API remains in very stable crystalline form in the dosage form, avoiding many of the physical stability problems associated with more expensive methods such as hot melt extrusion or spray drying.

In other embodiments, such as when a sustained release dosage form is desired, the process may be performed by feeding only the poorly soluble drug into an extruder, in the absence of a surfactant or other excipient, at a processing temperature that is just below the melting point of the drug.

In one aspect, the disclosed technology relates to a process for manufacturing an oral pharmaceutical dosage form including: (a) mixing a poorly soluble active pharmaceutical ingredient (API) and a surfactant into a blend; (b) feeding the blend into a processor that applies heat and shear forces to the blend at a processing temperature within a range of approximately the melting point of the surfactant to 3° C. below the melting point of the surfactant so as to form melt-coated, melt-granulated API granulates; and (c) formulating the API granulates into a sustained release oral pharmaceutical dosage form; wherein the API content in the dosage form is at least 60%, at least 70%, at least 80%, or at least 90% by weight, based on the total weight of the dosage form. In some embodiments, the processing temperature is ranges from the melting point of the surfactant to 2° C. below the melting point of the surfactant. In some embodiments, the processing temperature is approximately equal to the melting point of the surfactant. The actual processing temperature range can depend on properties that depend on the chemical nature of the API and the surfactant, including for example, the actual melting point of the API, whether the API is soluble in the molten surfactant, and if so, whether it forms a eutectic point, etc.

In some embodiments, the API is poorly soluble. In some embodiments, the API is an antibiotic, an anti-parasitic agent, an antiviral, an analgesic, an anti-cancer agent, an anti-inflamatory agent, or any other API that requires a dosing above 300 mg per unit dose. In some embodiments, the API is selected from favipiravir, ibuprofen, carbamazepine, fenofibrate, indomethacin, imatinib, flufenamic acid, erlotinib hydrochloride, vitamin D, estradiol, and combinations thereof.

In some embodiments, step (a) and/or step (c) further includes mixing at least one additional API into the blend. In some embodiments, the at least one additional API is selected from a steroid, an anti-inflammatory agent, a non-steroidal anti-inflammatory drug, an antibiotic, an antiviral agent, an anti-cancer agent, an analgesic, an anti-histaminic agent, and combinations thereof. In some embodiments, the surfactant includes one or more of poloxamer, polyoxyethylene stearate, cetylpyridinium chloride, polysorbate, and glyceryl monostearate. In some embodiments, step (a) further includes mixing into the blend one or more pharmaceutically acceptable excipients selected from controlled release agents, carriers, fillers, extenders, binders, humectants, disintegrants, absorption accelerators, wetting agents, absorbents, lubricants, coloring agents, and diluents. In some embodiments, step (c) includes combining the API granulates with one or more pharmaceutically acceptable excipients selected from controlled release agents, carriers, fillers, extenders, binders, humectants, disintegrants, absorption accelerators, wetting agents, absorbents, lubricants, coloring agents, and diluents. In practicing these and other embodiments of the disclosed technology, the skilled artisan can achieve desired attributes of the unit dose (dissolution, hardness, and the like) by selecting specific excipients and placing them inside the granules (i.e., in step (a) of the process disclosed herein); outside the granules (i.e., in step (c) of the process disclosed herein), or both.

In some embodiments, the one or more pharmaceutically acceptable excipients includes a controlled release agent. In some embodiments, the one or more pharmaceutically acceptable excipients includes one or more of hydroxypropyl methylcellulose, crospovidone, sodium carboxymethyl cellulose, and methyl cellulose. In some embodiments, the dosage form is selected from a tablet, a capsule, and a powder. In some embodiments, step (c) includes subjecting the API granulates to a compaction pressure to form a tablet, wherein the compaction pressure is greater than or equal to a pressure selected from 300 psi, 400 psi, 500 psi, 600 psi, 1000 psi, and 1800 psi.

In some embodiments, (i) steps (a) and (b) are performed as part of a continuous process, (ii) steps (b) and (c) are performed as part of a continuous process, or (iii) steps (a), (b), and (c) are performed as part of a continuous process. In some embodiments, the process is operated under closed loop control using a combination of sensors, controllers, and actuators to maintain the process within a desired range of operating parameters.

In another aspect, the disclosed technology relates to a dosage form prepared by a process disclosed herein.

In another aspect, the disclosed technology relates to an oral pharmaceutical dosage form including granulates of a poorly soluble API, wherein the dosage form has a total API content of at least 60 wt %, at least 70 wt %, at least 80 wt %, or at least 90 wt % and a surfactant content of less than or equal to 10 wt %, based on the total weight of the dosage form; and wherein the API granulates are capable of being directly compressed into tablets at a compaction pressure greater than or equal to a pressure selected from 300 psi, 400 psi, 500 psi, 600 psi, 1000 psi, and 1800 psi. In some embodiments, the dosage form is selected from a tablet, a capsule, and a powder. In some embodiments, the dosage form is a tablet having an average breaking force of more than 50 N, such as 55 N or more. In some embodiments, the dosage form has faster dissolution than a comparative dosage form that differs only by having been made from a physical mix of the API and surfactant, as determined by the time required to release 80% of the darunavir from the dosage form, when tested using any one of the following: (1) a USP II apparatus, 900 ml vessel, 75 RPM, using deionized water; (2) a USP II apparatus, 900 ml vessel, 75 RPM, using pH 2 simulated gastric fluid; or (3) a USP II apparatus, 900 ml vessel, 75 RPM, using pH 6.8 buffer.

In another aspect, the disclosed technology relates to a sustained release oral pharmaceutical dosage form including pure API granulates, wherein the dosage form: (i) has a total API content of at least 95 wt % based on the total weight of the dosage form; (ii) includes about 200 mg to about 800 mg total API; and (iii) has an average breaking force of at least 50 N, such as 55 N or more.

In another aspect, the disclosed technology relates to a process for manufacturing an immediate release darunavir-containing oral pharmaceutical dosage form, including: (a) mixing darunavir and a surfactant into a blend; (b) feeding the blend into a processor that applies heat and shear forces to the blend at a processing temperature within a range of approximately equal to the melting point of the surfactant to a temperature 3° C. below the melting point of the surfactant so as to form melt-coated, melt-granulated granulates; and (c) formulating the granulates into an immediate release oral pharmaceutical dosage form.

In some embodiments, the processing temperature ranges from approximately the melting point of the surfactant to 2° C. below the melting point of the surfactant. In some embodiments, In some embodiments, the processing temperature is approximately equal to the melting point of the surfactant. In some embodiments, In some embodiments, the surfactant includes one or more of poloxamer, polyoxyethylene stearate, cetylpyridinium chloride, polysorbate, and glyceryl monostearate. In some embodiments, In some embodiments, step (a) further includes mixing into the blend at least one additional active pharmaceutical ingredient (API) other than darunavir. In some embodiments, step (c) further includes mixing the darunavir granulates with at least one additional active pharmaceutical ingredient (API) other than darunavir. In some embodiments, the at least one additional API is selected from a steroid, an anti-inflammatory agent, a non-steroidal anti-inflammatory drug, an antibiotic, an antiviral agent, an anti-cancer agent, an analgesic, an anti-histaminic agent, and combinations thereof.

In some embodiments, step (a) further includes mixing into the blend one or more pharmaceutically acceptable excipients selected from controlled release agents, carriers, fillers, extenders, binders, humectants, disintegrants, absorption accelerators, wetting agents, absorbents, lubricants, coloring agents, and diluents. In some embodiments, step (c) further includes combining the granulates with one or more pharmaceutically acceptable excipients selected from controlled release agents, carriers, fillers, extenders, binders, humectants, disintegrants, absorption accelerators, wetting agents, absorbents, lubricants, coloring agents, and diluents. In some embodiments, the one or more pharmaceutically acceptable excipients includes a disintegrant, such as crospovidone, sodium carboxymethyl cellulose, methyl cellulose, or the like and combinations thereof. In some embodiments, both step (a) and step (c) comprise the inclusion of one or more disintegrants. While the overall attributes of the granules and the dosage form will depend on the nature and amount of excipients used in steps (a) and (c), a skilled artisan is able to select suitable materials in appropriate quantities.

In some embodiments, the dosage form is selected from a tablet, a capsule, and a powder. In some embodiments, step (c) includes subjecting the granulates to direct compression at a compaction pressure of greater than or equal to 600 psi to form a tablet. In some embodiments, (i) steps (a) and (b) are performed as part of a continuous process, (ii) steps (b) and (c) are performed as part of a continuous process, or (iii) steps (a), (b), and (c) are performed as part of a continuous process. In some embodiments, the processor is an extruder, blender, mixer, twin screw granulator, or kneader. In some embodiments, the process is operated under closed loop control using a combination of sensors, controllers, and actuators to maintain the process within a desired range of operating parameters.

In another aspect, the disclosed technology relates to an immediate release darunavir-containing oral pharmaceutical dosage form prepared by a process disclosed herein.

In another aspect, the disclosed technology relates to an immediate release darunavir-containing oral pharmaceutical dosage form including granulates including darunavir and surfactant, wherein the dosage form has a darunavir content of at least 75 wt % and a surfactant content of less than or equal to 10 wt %, based on the total weight of the dosage form; and wherein the granulates are capable of being directly compressed into tablets at a compaction pressure of greater than 300 psi, greater than 400 psi, greater than 500 psi, greater than 600 psi, greater than 1000 psi, or greater than 1800 psi. In some embodiments, the granulates further include at least one additional active pharmaceutical ingredient other than darunavir. In some embodiments, the at least one additional active pharmaceutical ingredient other than darunavir is added in step (c) as an extragranular ingredient. In some embodiments, the dosage form is selected from a tablet, a capsule, and a powder. In some embodiments, the dosage form is a tablet having an average breaking force of more than 50 N, such as 55 N or more.

In some embodiments, the dosage form has faster dissolution than a comparative dosage form that differs only by having been made from a physical mix of the darunavir and surfactant (i.e., without purposeful simultaneous application of heat and shear), as determined by the time required to release 80% of the darunavir from the dosage form, when tested using any one of the following: (1) a USP II apparatus, 900 ml vessel, 75 RPM, using deionized water; (2) a USP II apparatus, 900 ml vessel, 75 RPM, using pH 2 simulated gastric fluid; or (3) a USP II apparatus, 900 ml vessel, 75 RPM, using pH 6.8 buffer.

A variety of additional aspects will be set forth in the description that follows. The aspects can relate to individual features and to combinations of features. It is to be understood that both the foregoing general description and the following detailed description are exemplary and explanatory only and are not restrictive of the broad inventive concepts upon which the embodiments disclosed herein are based. The person of ordinary skill in the art, with a practical understanding of pharmaceutical formulations and pharmaceutical manufacturing, would know how to make and use the technology disclosed herein, to achieve additional variations in composition of matter, manufacturing process, and product formulation methods.

### BRIEF DESCRIPTION OF THE DRAWINGS

The accompanying drawings, which are incorporated herein and constitute part of this specification, are illustrative of particular embodiments of the present disclosure and do not limit the scope of the present disclosure. The drawings are not to scale and are intended for use in conjunction with the explanations in the following detailed description.

FIG. 1A is a graph showing the dissolution profiles of two physical mixtures and pure favipiravir, as described in Example 1. FIG. 1B is a photograph of the results of direct compression of a physical mixture of favipiravir and Poloxamer 188 (P188) at a compaction pressure of 1000 psi, as described in Example 1. FIG. 1C is a photograph of the results of direct compression of a physical mixture of favipiravir and P188 at a compaction pressure of 1800 psi, as described in Example 1.

FIG. 2 is a schematic illustration of the twin screw processor described in Example 1.

FIG. 3 is a collection of magnified images showing favipiravir and P188 before and after blending and treatment in a twin screw granulator (processor, TSG) applying heat and shear at a processing temperature of 45° C. or 55° C., as described in Example 1.

FIG. 4 is a graph showing the dissolution profiles of a physical mixture, a treated blend of favipiravir and P188 processed at 45° C., a treated blend of favipiravir and P188 processed at 55° C., and pure favipiravir, as described in Example 1.

FIG. 5 is a collection of photographs showing tablets compressed from a treated blend of favipiravir and P188 processed at 55° C. at compaction pressures of 600 psi, 800 psi, and 1000 psi, as described in Example 1. The photographs on the left show side views of the tablets; the photographs in the center show top views of the tablets; and the photographs on the right show the result of crushing hardness testing of the tablets.

FIG. 6 is a graph showing dissolution profiles of granulates, including immediate release favipiravir granulates as described in Example 2.

FIG. 7 is a graph showing dissolution profiles of tablets, including sustained release favipiravir tablets, prepared and tested as described in Example 3.

FIG. 8 shows a schematic of the screw configuration of a twin-screw extruder described in Example 4.

FIG. 9 is a graph showing the dissolution profile of darunavir tablets as described in Example 5.

FIG. 10A is a graph showing the dissolution profile of granules containing 91.5 wt % darunavir as described in Example 5. FIG. 10B is a graph showing the dissolution profile of granules containing 86.5 wt % darunavir as described in Example 5.

FIG. 11 is a graph showing the dissolution profile of granules containing darunavir, P188, LMW HPMC, HMW HPMC, and Lactose, and the dissolution profile of tablets made from this blend after lubricating the granules with MgSt.

FIG. 12 is a graph showing the dissolution profile of tablets containing different amounts of intragranular (Int) and extra-granular (Ext) disintegrant and other excipients, as per the compositions in Table 12.

FIG. 13A, FIG. 13B, and FIG. 13C are a collection of graphs showing the dissolution profile of granules containing 69.6% darunavir, 10% P188, 3% Crospovidone, and 17% of Chlorpheniramine Maleate (CPM), Ibuprofen (Ibu), and Dexamethasone (Dexa), respectively.

FIG. 14A is a graph showing that the presence of CPM inside the darunavir granules results in tablets with significantly slower dissolution. FIG. 14B is a graph showing that when CPM is placed outside the darunavir granules, the resulting tablet containing about 85 wt % API exhibits immediate release of darunavir. FIG. 14C is a graph showing that when Ibu is placed outside the darunavir granules, the resulting tablet containing about 85 wt % API exhibits immediate release of darunavir.

### DETAILED DESCRIPTION

The following discussion omits or only briefly describes conventional features of the disclosed technology that are apparent to those skilled in the art. Reference to various embodiments does not limit the scope of the claims attached hereto. Additionally, any examples set forth in this specification are intended to be non-limiting and merely set forth some of the many possible embodiments for the appended claims. Further, particular features described herein can be used in combination with other described features in each of the various possible combinations and permutations. A person of ordinary skill in the art would know how to use the instant invention, in combination with routine experiments, to achieve other outcomes not specifically disclosed in the examples or the embodiments.

Unless otherwise specifically defined herein, all terms are to be given their broadest possible interpretation including meanings implied from the specification as well as meanings understood by those skilled in the art and/or as defined in dictionaries, treatises, etc. Unless defined otherwise, all technical and scientific terms used herein have the same meaning as commonly understood by one of ordinary skill in the art in the field of the disclosed technology. It must also be noted that, as used in the specification and the appended claims, the singular forms “a,” “an” and “the” include plural referents unless otherwise specified, and that the terms “includes” and/or “including,” when used in this specification, specify the presence of stated features, elements, and/or components, but do not preclude the presence or addition of one or more other features, steps, operations, elements, components, and/or groups thereof. Additionally, methods, equipment, and materials similar or equivalent to those described herein can also be used in the practice or testing of the disclosed technology.

### API and API Granulates

As used herein, the term “API granulates” or “API granules” refers to agglomerates of particles of one or more active pharmaceutical ingredient (API). For API granulates formed from API and surfactant, this term refers to agglomerates of particles of one or more API having at least a partial coating of surfactant that has been melted onto the outer surfaces of the API particles according to the simultaneous melt-granulation and melt-coating process disclosed herein. For API granulates formed from pure API in the absence of a surfactant or other excipient (also referred to as “pure API granulates”), this term refers to agglomerated particles of one or more API in the absence of any excipients.

Any suitable, pharmaceutically acceptable drug or pro-drug substance may be used in connection with the disclosed technology. Further, one or more different APIs (e.g., 1, 2, 3 or more APIs) may be processed with one or more different substances having similar functions (e.g., 1, 2, 3 or more surfactants) or having different functions (e.g., a surfactant, a chemical stabilizer, and a taste masking ingredient). Moreover, it is also understood and known in the field that a given substance can have multiple functions, and those functions might be expressed to different extents under various situations. Examples of substances with multiple functions include magnesium stearate (MgSt) (a lubricant, a glidant, and a hydrophobic material), hydroxypropyl methylcellulose (HPMC) (a binder, a wetting agent, and a controlled release agent), many forms of starch (wet binders compression binders, fillers, and disintegrants), etc.

Specific APIs disclosed herein are provided for illustrative purposes only and do not limit the scope of the disclosed technology. In general, the APIs used in the disclosed process should be chemically and physically stable under relevant experimental conditions, and soluble to a significant extent in different types of solvents. In some embodiments, the API is suitably soluble in a volatile organic solvent. In general, the melting point of at least one API should be higher than the melting point of at least one surfactant. In some embodiments, the API is suitably soluble in water. In other embodiments, the API is poorly soluble in water—e.g., API solubility is less than 10 mg/ml. Non-limiting examples of APIs that may be used in connection with the disclosed technology include darunavir, favipiravir, ibuprofen, carbamazepine, fenofibrate, indomethacin, flufenamic acid, imatinib, erlotinib hydrochloride, vitamin D, steroids, estradiol, other non-steroidal anti-inflammatory drugs (NSAIDs), antibiotics, antivirals (e.g., anti-HIV drugs), and combinations thereof. In some embodiments, the API is darunavir. In some embodiments, the API is favipiravir. In some embodiments, the API has an average particle size of less than 100 micrometers, such as 30-60 micrometers. In some embodiments, the API granulate does not contain darunavir. In some embodiments, at least one additional API is present in the formulation.

Darunavir (molecular formula: C₂₇H₃₇N₃O₇S) is a poorly soluble API with a solubility of approximately 8.7 mg/L in water at 25° C. The melting point of darunavir is 74-76° C.

Specific surfactants disclosed herein are provided for illustrative purposes only and do not limit the scope of the disclosed technology. Any suitable, pharmaceutically acceptable surfactant(s) may be used in connection with the disclosed technology so long as the melting point of the surfactant is lower than the melting point of the API with which it is to be combined, and neither the surfactant nor the API experience substantial degradation when exposed to the temperatures and shear rates required to achieve simultaneous melt-granulation and melt-coating. In some embodiments, the melting point of the surfactant is at least 10° C. lower, at least 15° C. lower, at least 20° C. lower, or at least 25° C. lower than the melting point of the API. In some embodiments, the difference between the melting point of the surfactant and the comparatively higher melting point of the API is 15° C. to 150° C., 20° C. to 150° C., 15° C. to 100° C., or 20° C. to 125° C.

The surfactant may be amphoteric, non-ionic, cationic or anionic. Non-limiting examples of suitable surfactants include: cetylpyridinium chloride, sodium lauryl sulfate, monooleate, sorbitan monooleate, monolaurate, monopalmitate, monostearate or another ester of polyoxyethylene sorbitane or polyethylene glycol, diethylene glycol monostearate, glyceryl monostearate, sodium dioctylsulfosuccinate, lecithin, stearylic alcohol, cetostearylic alcohol, cholesterol, polyoxyethylene ricin oil, macrogolglycerol ricinoleate, macrogolglycerol hydroxystearate, polyoxyl 35 castor oil, polyoxyl castor oil such as polyoxyl 40 hydrogenated castor oil, hydrogenated polyoxyethylene fatty acid glycerides, pluronic surfactants such as poloxamers of different molecular weights (e.g., poloxamer 124, poloxamer 188, poloxamer 237, poloxamer 338, poloxamer 407), block copolymers of poly(ethylene oxide) and poly(propylene oxide), inulin, inutec, benzethanium chloride, docusate sodium, polyoxyethylene sorbitan fatty acid esters, polysorbate (e.g., polysorbate 80, polysorbate 60), vitamin E derivatives, polyoxyethylene alkyl ethers, polyoxyethylene stearates, saturated polyglycolyzed glycerides, fatty amine oxides, fatty acid alkanolamides, poly(oxyethylene)-block-poly(oxypropylene) copolymers, and combinations thereof.

Poloxamer is available in various grades, as indicated in Table 1 below based on its chemical structure:

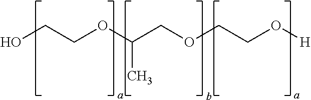

TABLE 1

| Poloxamer | Physical Form | a | b | Average molecular weight |
|---|---|---|---|---|
| 124 | Liquid | 12 | 20 | 2090-2360 |
| 188 | Solid | 80 | 27 | 7680-9510 |
| 237 | Solid | 64 | 37 | 6840-8830 |
| 338 | Solid | 141 | 44 | 12700-17400 |
| 407 | Solid | 101 | 56 | 9840-14600 |

In some embodiments, the API granulates disclosed herein include at least 90 wt % API, at least 95 wt % API, at least 96 wt % API, at least 97 wt % API, at least 98 wt % API, or at least 99 wt % API, based on the total weight of the API granulate.

In some embodiments, the API granulates disclosed herein have a surfactant content of less than or equal to 10 wt %, less than or equal to 9 wt %, less than or equal to 8 wt %, less than or equal to 7 wt %, less than or equal to 6 wt %, less than or equal to 5 wt %, less than or equal to 4 wt %, less than or equal to 3 wt %, less than or equal to 2 wt %, less than or equal to 1 wt %, based on the total weight of the API granulate.

In some embodiments, the API granulates disclosed herein have an average diameter of about 100 micrometers to about 1000 micrometers, such as about 200 micrometers to about 800 micrometers. In some embodiments, the API granulates disclosed herein have an average diameter that is at least three times (3×) greater (e.g., 4× or more, or 5× or more)) than the average particle size of the largest API present in the formulation.

### Pharmaceutical Dosage Forms

One or more surfactants can advantageously increase the rate of dissolution of the API granulates by facilitating wetting, and can also increase the maximum drug concentration of a finished pharmaceutical drug product produced therefrom by eliminating the need to transform the API into an amorphous solid dispersion, i.e., by spray drying, hot melt extrusion, or other techniques that disperse API molecules within a matrix of another substance. In general, pharmaceutical dosage forms formulated from API granulates disclosed herein are more soluble than their counterparts made from ingredients that were physically mixed without simultaneous application of shear and heat at a processing temperature very close to the melting point of the surfactant.

In some embodiments, pharmaceutical dosage forms formulated from surfactant-containing API granulates disclosed herein have an API content of at least 80 wt %, at least 85 wt %, at least 90 wt %, or at least 95 wt % based on the total weight of the pharmaceutical dosage form. In one embodiment, the pharmaceutical dosage form contains 200 mg to 2000 mg of the API (e.g., favipiravir, darunavir, or any other API listed herein, optionally in combination with one or more other APIs). In another embodiment, the pharmaceutical dosage form contains 200 mg, 400 mg, 600 mg, 800 mg, 1000 mg, or 1200 mg of the API (e.g., favipiravir, darunavir, or any other API listed herein, optionally in combination with one or more other APIs).

In some embodiments, pharmaceutical dosage forms formulated from API granulates disclosed herein have a surfactant content of less than or equal to 10 wt %, less than or equal to 9 wt %, less than or equal to 8 wt %, less than or equal to 7 wt %, less than or equal to 6 wt %, less than or equal to 5 wt %, less than or equal to 4 wt %, less than or equal to 3 wt %, less than or equal to 2 wt %, less than or equal to 1 wt %, based on the total weight of the pharmaceutical dosage form.

In some embodiments, pharmaceutical dosage forms formulated from API granulates disclosed herein have a surfactant content of about 1-10 wt %, such as about 1-8 wt %, about 1-6 wt %, about 1-5 wt %, about 1-4 wt %, about 1-3 wt %, about 2-10 wt %, about 2-8 wt %, about 2-6 wt %, about 2-5 wt %, about 2-4 wt %, about 3-10 wt %, about 3-8 wt %, about 3-6 wt %, about 3-5 wt %, about 4-10 wt %, about 4-8 wt %, about 4-6 wt %, about 2-10 wt %, about 5-10 wt %, about 6-10 wt %, about 7-10 wt %, or about 8-10 wt %, based on the total weight of the API in the pharmaceutical dosage form.

In some embodiments, pharmaceutical dosage forms formulated from API granulates disclosed herein have a total excipient content of less than 20 wt %, less than 15 wt %, less than 10 wt %, or less than 5 wt %, based on the total weight of the pharmaceutical dosage form.

In other embodiments, pharmaceutical dosage forms formulated from pure API granulates disclosed herein have an API content of at least 95 wt %, at least 96 wt %, at least 97 wt %, at least 98 wt %, or at least 99 wt % based on the total weight of the pharmaceutical dosage form. In one embodiment, the pharmaceutical dosage form contains about 200 mg to about 700 mg of API, such as about 300 mg to about 600 mg. In another embodiment, the pharmaceutical dosage form contains 200 mg, 300 mg, 400 mg, 500 mg, 600 mg, or 700 mg of the API. In some embodiments, pharmaceutical dosage forms formulated from pure API granulates disclosed herein have a total excipient content of 5 wt % or less, 4 wt % or less, 3 wt % or less, 2 wt % or less, or 1 wt % or less, based on the total weight of the pharmaceutical dosage form. In some embodiments, pharmaceutical dosage forms formulated from pure API granulates have an average breaking force (hardness) of at least 50 N, such as at least 60 N, at least 70 N, at least 80 N, at least 90 N, at least 100 N, at least 110 N, or at least 120 N.

As disclosed herein, finished pharmaceutical drug products, such as solid oral dosage form pharmaceutical compositions, are suitable for administration to a subject, such as a human or other mammal. Non-limiting examples of solid oral dosage forms include tablets, capsules containing API granulates as described herein, optionally with other ingredients, capsules including a plurality of mini-tablets, powders, and granulations, and other dosage forms that are manufactured from powders and granulates. Non-limiting examples of tablets include sublingual molded tablets, buccal molded tablets, sintered tablets, compressed tablets, chewable tablets, freeze-dried tablets, soluble effervescent tablets, and pellets. Non-limiting examples of capsules, in which a solid dosage form of the drug is enclosed within a hard or soft soluble container or shell, include hard gelatin capsules, soft gelatin capsules, and non-gelatin capsules. In some embodiments, the finished solid oral dosage form may be modified to achieve a desired timing of API release—e.g., a dosage form that provides immediate release, sustained release, controlled release, extended release, partial immediate and partial delayed release, and combinations thereof.

In some embodiments, the finished solid oral dosage form is an immediate release darunavir-containing oral pharmaceutical dosage form. In other embodiments, the finished solid oral dosage form is a sustained release API-containing (e.g., favipiravir-containing) oral pharmaceutical dosage form. In some embodiments, the sustained release API-containing oral pharmaceutical dosage form does not contain darunavir.

In some embodiments, finished pharmaceutical drug products prepared by the disclosed process provide sustained release, whereby API is released from the drug product over a period of time, such as up to 24 hours, up to 22 hours, up to 20 hours, up to 18 hours, up to 16 hours, up to 14 hours, up to 12 hours, up to 10 hours, or up to 8 hours. In one embodiment, the finished dosage form releases API for up to 12 hours.

The disclosed process can also be used in the manufacture of non-oral products where a mixture of APIs and other solid ingredients is useful, including but not limited to the manufacture of inhalants, implantable and injectable solid compositions, vascular stents, ocular implants, and the like.

### Excipients

In preparing a pharmaceutical drug product, including a finished solid oral dosage form, the API granulates may be blended with one or more pharmaceutically acceptable excipients. Non-limiting examples of such excipients include: carriers, such as cellulose or substituted cellulose materials, sodium citrate or dicalcium phosphate; fillers or extenders, such as starch-based materials, microcrystalline cellulose, lactose, sucrose, glucose, mannitol, and/or silicic acid; binders, such as hydroxypropyl cellulose, hydroxypropyl methylcellulose, carboxymethyl-cellulose, alginates, gelatin, polyvinyl pyrrolidone, sucrose and/or acacia; humectants, such as glycerol; disintegrants, such as crospovidone, sodium carboxymethyl cellulose, agar-agar, calcium carbonate, potato or tapioca starch, alginic acid, certain silicates, and sodium carbonate; absorption accelerators, such as quaternary ammonium compounds and additional surfactants, such as poloxamer and sodium lauryl sulfate; wetting agents, such as cetyl alcohol, glycerol monostearate, and non-ionic surfactants; absorbents, such as kaolin and bentonite clay; lubricants, such as talc, calcium stearate, magnesium stearate, solid polyethylene glycols, sodium lauryl sulfate, zinc stearate, sodium stearate, stearic acid, and mixtures thereof; coloring agents; controlled release agents, such as ethyl cellulose, poly(ethylene oxide), alkyl-substituted celluloses, crosslinked polyacrylic acids, xanthan gum, guar gum, carrageenan gum, locust bean gum, gellan gum, methyl cellulose, hydroxypropyl methyl cellulose, hydroxyethyl cellulose, ethyl cellulose, sodium alginate, gelatin, modified starches, co-polymers of carboxyvinyl polymers, co-polymer of acrylates, co-polymers of oxyethylene and oxypropylene and mixtures thereof; diluents; and other additives, such as paraffin and high molecular weight polyethylene glycols. In mentioning these materials, the use of the term “such us” means that the mentioned materials are simply examples belonging to a more extensive class. Also, as it is known in the art, most pharmaceutical ingredients have more than one useful function, and the mention of any one ingredient in any one of the above examples is not meant to exclude the use of that material for a different purpose. For example, as mentioned, starch can be a filler, a binder, and a disintegrant, and many other materials can also be used in more than one way.

In some embodiments, the API granulates are blended with a small amount of one or more excipients that impact or control the release of the API. For example, such excipients may include a sustained release agent such as HPMC (e.g., high molecular weight HPMC) to facilitate extended release, or a disintegrant such as CMC-Na to facilitate immediate release. In some embodiments, the amount of such an excipient (e.g., a sustained or controlled release agent or an immediate release agent) in the finished pharmaceutical dosage form is less than or equal to 6 wt %, less than or equal to 5 wt %, less than or equal to 4 wt %, or less than or equal to 3 wt %, based on the total weight of the pharmaceutical dosage form.

In some embodiments, the API granulates are blended with a small amount of lubricant, such as magnesium stearate (MgSt). In some embodiments, the amount of lubricant in the finished pharmaceutical dosage form is less than or equal to 1 wt %, less than or equal to 0.7 wt %, less than or equal to 0.5 wt %, or less than or equal to 0.3 wt %, based on the total weight of the pharmaceutical dosage form.

In some embodiments, the disclosed process eliminates several expensive excipients from the formulation of the finished dosage form, thus advantageously lowering cost and eliminating sources of variability that can cause quality problems in finished products.

### Simultaneous Melt-Granulating/Melt-Coating Process

In general, the simultaneous melt-granulating and melt-coating process disclosed herein includes the steps of: combining particles of at least one API with at least one surfactant to form a pre-mixed blend, and subjecting the blend to simultaneous shear and heat in order to form API granulates. In some embodiments, on average, the majority of each API particle is coated with surfactant. Not wishing to be bound by any particular theory, it is nonetheless believed that the simultaneous application of heat and shear causes localized melting of the particles of the low melting point substance at points where these particles are in direct contact with API particles. This promotes adhesion of the low melting point surfactant to the surface of the API particles as well as adhesion and agglomeration among surfactant-coated API particles.

The presence of surfactant particles on and among the surfaces of the API particles makes the combined particles easier to wet. The API granulates thus produced (“treated”) exhibit substantially enhanced dissolution as compared to untreated API particles (i.e., API alone), and also as compared to a physical mix of the same API and the same proportion of the same surfactant. As used herein, the term “physical mix” or “physical mixture” refers to a combination of API(s) and surfactant(s) that has not been subjected to shear and heat very close to the melting point of the surfactant according to the process disclosed herein.

Importantly to the invention disclosed here, there exists an optimum temperature range that maximizes the enhancement of dissolution of a poorly soluble API. When the process is conducted at temperatures close to the melting point of the surfactant, the process generated larger, friable granules with low density and good flow properties. Such granules, and tablets made with such granules, generally demonstrate very large enhancement in dissolution properties. If the process is conducted at temperatures more than 10° C. below the melting point of the surfactant, only small, poorly flowable granules are typically obtained. Finally, when the process is conducted at temperatures significantly above the melting point of the surfactant, it produces large dense granules. Such granules, and tablets made with such granules, generally demonstrate much smaller enhancement of dissolution properties.

Surprisingly, the coating and granulation processes can be managed independently, to maximize the extent of coating and control the type of granules generated. For the enhancement of dissolution, the process must be conducted in a manner that increases the extent of coating, while generating lower density granules. The skilled artisan is able to determine such conditions (process temperature, mass flow rate, speed of the screws in the processor). While not wishing to be bound by theory, a reasonable explanation is that if the process is conducted at barrel temperatures that are too high, extensive melting of the surfactant (or any other melt binder) occurs. API particles are then able to slide past each other, lubricated by the molten surfactant/binder. Since the twin screw processor necessarily applies significant compressive stress to the mixture, a system where the surfactant/binder has been extensively melted results in denser granules that dissolve more slowly. Conversely, when conditions are selected to enable the instant invention, only a small fraction of the surfactant will experience localized melting due to the simultaneous effects of heat and shear, causing the small amount of melted surfactant to coat the API particles due to the shearing of the material, but avoiding the dense packing of particles that would prevent enhancement of dissolution.

The enhanced dissolution properties of the disclosed API granulates is particularly surprising because it was previously thought that increasing an extruder barrel temperature to the melting point of surfactant would not yield optimal dissolution, and would instead cause a slightly delayed dissolution in comparison to untreated powder. Especially at the beginning of the dissolution (<100 min), a smaller amount of drug molecules could be released at each sampling point. Such behavior can be interpreted as the formation of hard granulates (or pellets) when the surfactant is completely melted. The formation of hard granulates would prevent the dissolution medium from penetrating into the matrix of granulates, which would thus reduce the total area of surface that is fully exposed to the dissolution medium. However, as disclosed herein, it was unexpectedly found that very rapid simultaneous coating and granulation at temperatures close to the surfactant melting point is highly advantageous and greatly enhances dissolution.

Additionally, continuous manufacturing, in contrast to traditional batch processing, allows for the manufacturing of drug products from raw materials in a single continuous fashion such that the output is maintained at a consistent rate with no need to stop production. As a result, the disclosed technology is capable of efficiently providing homogenous pharmaceutical drug products containing large amounts of API in a robust, readily controlled, and commercially valuable manufacturing process.

In the disclosed process, melt-granulating and melt-coating can be performed continuously, either as a stand-alone process for continuously manufacturing API granulates, or as part of a larger integrated continuous manufacturing line for manufacturing pharmaceutical dosage forms containing the API granulates. Continuous manufacturing methods can provide significant technical and business advantages relative to batch methods. In general, continuous manufacturing methods are more robust and controllable. They achieve the same production rates as batch processes in much smaller and thus less capital-intensive equipment, which also requires less space to operate. They also facilitate automation that can be used to achieve significant improvements in product quality and process reliability. By combining melt-granulating, melt-coating and continuous manufacturing, the disclosed process achieves benefits afforded by both technologies and may be used as a rapid development platform to prepare clinical supplies and to introduce new drugs to market, or to manufacture those products at higher qualities and lower cost.

Further, the disclosed process allows for an integrated technology for continuous melt-granulating and melt-coating that is designed and optimized based on a deeper understanding of the main components of the manufacturing system, helping to promote adoption of modern methodologies across an essential industry that at the present time often uses empirical methods and batch processes as its main development and manufacturing paradigm. The continuous manufacturing processes described herein may include sensing and control capabilities, such that the process is continuously monitored by various sensors, controllers, and actuators to maintain the continuous process and the resulting products within the desirable operating range of process parameters and product quality attributes. Measurements collected from sensors can be used in conjunction with controllers and actuators arranged in a closed loop system, using feedback, feed forward, and other configurations to control the performance of the process and the quality of the manufactured products.

The disclosed process also provides one or more significant advantages that make it very useful as a commercial method for drug product development and manufacturing. For example, the process is seemingly easy to perform, whereby a finished drug product may be made by melt-granulating and melt-coating surfactant(s) onto API particles to produce API granulates, optionally mixing the API granulates with other ingredients, including other APIs, compacting the API granulates into tablets or filling them into capsules, vials, blister packs, or the like. This process can eliminate expensive processing steps, such as crystallization, drying, and milling of the drug material, batch blending with excipients, and the like. By simplifying the process, the method significantly accelerates product development.

The disclosed process is also readily up-or down-scalable, facilitating manufacturing at both clinical trial and commercial scales and enabling rapid scale-up (or scale-down) and scale-out of manufacturing rates to meet changing market demands. In its simplest form, a continuous process enables the operator to make as much, or as little product as desired simply by changing the length of time the process is operated.

In some embodiments, the disclosed technology relates to processes of continuously manufacturing a finished pharmaceutical drug product using melt-coated API granulates made by either a continuous or a non-continuous (e.g., batch) process. In such embodiments, the material being processed in the continuous process flows through multiple simultaneous unit operations, including feeding API granulates into feeder, optionally combining the API granulates with one or more pharmaceutically acceptable excipients in a continuous processor, and compounding the mixture into a desired solid oral dosage form. Non-limiting examples of other suitable finishing steps include filling the mixture into capsules, vials, or aerosol blisters, or compressing the mixture into tablets.

The disclosed continuous manufacturing process may include a series of unit operations and online testing equipment. In one embodiment, the process includes a first feeder for delivering API, a second feeder for delivering surfactant, an optional blender for pre-blending API and surfactant, an optional mill for milling either API alone or pre-blended API and surfactant, and a processor capable of subjecting the API and surfactant to heat and shear simultaneously at a processing temperature that is very close to the melting point of the surfactant and achieves both melt-granulation and melt-coating of the API particles.

In one embodiment of the process, a first feeder dispenses API particles into a batch vessel such as a blender, and a second feeder dispenses surfactant into the same batch vessel. In some embodiments in which more than one type of API and/or more than one type of surfactant is used, additional corresponding feeders and/or blenders may also be employed. Alternatively, the first feeder may contain more than one type of API and/or the second feeder may contain more than one type of surfactant. The pre-mixed API and surfactant blend is then fed into a processor. In some embodiments, one or more excipients (e.g., one or more binders and/or disintegrants) are fed into the batch vessel with the API(s) and surfactant(s) to form a multi-component pre-mixed blend.

In some embodiments, the API and surfactant are fed (optionally, continuously fed) into the processor in the absence of a solvent. In some embodiments, the one or more API and one or more surfactant are fed (optionally, continuously fed) into the processor in the absence of a solvent and/or in the absence of any other materials (e.g., excipients, such as carriers, fillers, extenders, binders, humectants, disintegrants, absorption accelerators, wetting agents, absorbents, lubricants, coloring agents, and diluents). In some embodiments, the API feeder and/or the surfactant feeder is operated at room temperature. In some embodiments, more than one thermal processor is used as part of an integrated (optionally, continuous) manufacturing process.

In some embodiments, the API and surfactant (and other excipient(s), if present) may be directed into a batch vessel for pre-blending, after which the pre-blend may be directed into the processor. In some embodiments, the API or the optionally prepared pre-blend may be milled, and then directed into the processor.

In the processor, API and surfactant are subjected simultaneously to application of moderate heat and mechanical shear stress and compressive forces. As used herein, “shear stress” refers to a stress in a material in multiple directions, both parallel and orthogonal to the tangent to the surface of the API particles. In general, the shear stresses applied in the processor are converted to additional heat through friction and compression, which helps to melt or soften the surfactant without melting or otherwise physically or chemically modifying the API.

The processor parameters are selected so as to efficiently provide a melt-coating of surfactant on the outer surface of the API particles while simultaneously forming agglomerates by melt-granulation. In some embodiments, the majority of the outer surface of the API particles is coated with surfactant(s). In some embodiments, the surfactant coats 30% or more of the outer surface of the API particles, as determined by SEM images.

Non-limiting examples of suitable processors include extruders, such as single screw extruders and twin-screw extruders, blenders, mixers, kneaders, and other shearing devices. A heat source, such as a heat exchanger, may be provided as an integrally formed part of the processor. Optionally, heat may be included in the process as a separate device immediately prior to the application of shear. In one embodiment, the processor includes a heated barrel or jacket. In one embodiment, the processor includes an extruder having a single screw or multiple screws. When an extruder contains multiple screws, the screws may be arranged for co-rotation and/or counter-rotation. In some embodiments, the processor includes a combination of kneading and conveying elements (e.g., alternating kneading and conveying elements) wherein the kneading elements apply shear forces and the conveying elements transfer the API, surfactant, and API granulates through the processor. In some embodiments, the processor includes a rotating shearing device, such as a screw extruder, impeller, agitator, blade, or the like. The rotating shearing device may rotate at a speed of 100 rpm to 1000 rpm, such as 100 rpm to 700 rpm, 150 rpm to 500 rpm, or 150 rpm to 300 rpm.

In some embodiments, the processor may be a twin-screw granulator or twin screw extruder. In such instances, the processor may have a combination of kneading (K) zones and conveying (C) zones, which may be arranged in various patterns. In some embodiments, the zones alternate between C and K, with C zones on each end. For example, the processor may include the following sequence of zones: C—K—C—K—C—K—C—K—C or C—K—C—K—C—K—C. In some embodiments, the processor may have 1, 2, 3, 4, 5, or more kneading zones, and 2, 3, 4, 5, 6, or more conveying zones. In general, kneading zones include kneading blocks or elements; and conveying zones include conveying screws or elements. In some embodiments, a kneading zone has a plurality of kneading blocks—e.g., 2, 3, or 4 kneading blocks in any one or more kneading zone.

The processor may be operated in a selected mode, such as a partially filled or starved mode, which can be adjusted by adjusting the rate(s) at which the mixture of API and surfactant are fed into the processor. In a partially filed mode, a continuous volume of API, surfactant, and API granulates is maintained inside the processor during the process, wherein the continuous volume is 25-75% of the total volume of the processor. In a starved mode, the API and surfactant are fed into the processor at a slower rate than the rate at which the API granulates are formed. In some embodiments, the API and surfactant are fed into the processor at a rate of 100 g/h to 50 kg/h, such as 150 g/h to 1 kg/h, or 200 g/h to 300 g/h. In one embodiment, the API and the surfactant are not premixed, but they are added at a carefully controlled relative rate, and mixing happens within the extruder.

Processing Temperature: The API and surfactant are processed in the processor at a temperature close to the melting point of the surfactant and below the melting point of the API(s). When more than one API is used, the processing temperature is below the lowest melting point of the APIs. When more than one surfactant is used, the processing temperature is close to the lowest melting point of the surfactants. When the melting point of an API or surfactant is defined in terms of a range of temperature values, the average process temperature value should be relied upon for purposes of making and using the disclosed technology. The processing temperature is carefully selected so as to only partially melt or soften the surfactant, thus causing the surfactant to effectively smear onto the outer surfaces of the API particles, while simultaneously causing agglomeration, whereby the process results in the production of low density API granulates that have internal and external surfaces extensively coated with surfactant (as determined, for example, by SEM, EDX, and other related techniques known in the art). Temperatures outside the disclosed range (i.e., more than +10 degrees from the melting point of the surfactant) should be avoided.

In some embodiments, the processing temperature is at or below the melting point of the surfactant. For example, the processing temperature may range from approximately the melting point of the surfactant to a temperature that is 3° C., 2° C., or 1° C. below the melting point of the surfactant. As used herein, a reference to “approximately the melting point” of the surfactant refers to the melting point±10%.

Processing temperatures ranging between any of the foregoing temperatures are contemplated herein as well. For example, the processing temperature may range from: 3° C. below the melting point of the surfactant to approximately the melting point of the surfactant; 2° C. below the melting point of the surfactant to approximately the melting point of the surfactant; or 1° C. below the melting point of the surfactant to approximately the melting point of the surfactant.

The disclosed technology also contemplates the use of a temperature profile along the process, where, for example, a lower processor temperature may be used near the entrance of the materials to enable their homogenization prior to melting and coating, a higher temperature may then be applied in a central region of the processor to achieve the desired degree of coating, and a lower temperature might be used near the exit of the processor to cool down the coated particles and prevent them from sticking to one another or to equipment surfaces.

The time spent by the API or a detectable tracer material inside the processor during the disclosed continuous melt-coating process is the mean residence time (MRT). It has been surprisingly discovered that the application of heat and shear in the processor simultaneously enables substantial coating and agglomeration of the API particles with surfactant in very short processing times. The brief exposure to heat and shear substantially prevents thermal degradation of the API and/or the surfactant that would likely occur in batch processes requiring much longer times or higher temperatures to achieve comparable degrees of melt coating. In some embodiments, the MRT is less than or equal to 30 seconds, less than or equal to 1 minute, less than or equal to 2 minutes, less than or equal to 3 minutes, less than or equal to 4 minutes, less than or equal to 5 minutes, less than or equal to 6 minutes, less than or equal to 7 minutes, less than or equal to 8 minutes, less than or equal to 9 minutes, less than or equal to 10 minutes, or less than or equal to 15 minutes. In some embodiments, the MRT is 1 second to 30 seconds, 1 second to 1 minute, 1 second to 2 minutes, 1 second to 3 minutes, 1 second to 4 minutes, 1 second to 5 minutes, 1 second to 6 minutes, 1 second to 7 minutes, 1 second to 8 minutes, 1 second to 9 minutes, 1 second to 10 minutes, 30 seconds to 1 minute, 30 sec to 2 minutes, 30 sec to 3 minutes, 30 sec to 4 minutes, 30 seconds to 5 minutes, or 30 seconds to 10 minutes.

Formulation of API Granulates: After processing, the API granulates are collected from the processor. Subsequently, the collected API granulates may then be formulated into a finished drug product. Formulations may include the addition of small amounts of one or more excipients as discussed above. In some embodiments, formulation processing includes milling the API granulates and optional excipients. In some embodiments, formulation processing includes compression of API granulates and optional excipients into tablets, or filling into capsules or sachets or bottles. In some embodiments, extragranular components, such as controlled release agents, are combined with the API granulates in order to achieve a desired release profile for the finished pharmaceutical dosage form.

Non-limiting examples of finished drug products include solid oral dosage forms such as tablets, capsules, and powders. The finished drug product may be further provided in appropriate packaging, such as but not limited to a blister pack, a bottle, or vial.

Enhanced Properties: API granulates produced by the disclosed process have significantly enhanced properties, including dissolution, compactability, and flowability.

With respect to dissolution, dissolution testing may be performed to determine the release drug profile of the API granulates and also of finished dosage forms formulated from the API granulates. Among other devices suitable for dissolution testing known to those of ordinary skill in the art, a 708-DS, 8-spindle, 8-vessel USP dissolution apparatus type II (paddle), with automated online UV-Vis measurement (Agilent Technologies) could be used for such measurements. In some embodiments, the API granulates and finished drug products formulated therefrom may have a dissolution drug release profile such as, but not limited to:

An immediate release profile, where either the API (e.g., darunavir) granulates or the finished drug product formulated from the API granulates releases at least 80% of the API within 60 minutes, at least 80% of the API in less than 45 minutes, at least 80% of the API in less than 30 minutes, at least 80% of the API in less than 15 minutes, at least 80% of the API in less than 10 minutes, when tested in a USP II apparatus using 900 ml of simulated gastric fluid with pH<2, 50 RPM, and 37° C.

An immediate release profile, where either the API (e.g., darunavir) granulates or the finished drug product formulated from the API granulates releases at least 80% of the API within 60 minutes, at least 80% of the API in less than 45 minutes, at least 80% of the API in less than 30 minutes, at least 80% of the API in less than 15 minutes, at least 80% of the API in less than 10 minutes, when tested in a USP II apparatus using 900 ml of de-ionized water, 50 RPM, and 37° C.

A sustained release profile, where either the API granulates or the finished drug product formulated from the API granulates releases less than 80% of the API after 60 minutes, or after 120 minutes, or after 240 minutes, or after 360 minutes, or after 480 minutes, or after 1440 minutes, when tested in a USP II apparatus using 900 ml of de-ionized water, 50 RPM, and 37° C.

A sustained release profile, where either the API granulates or the finished drug product formulated from the API granulates releases less than 80% of the API after 60 minutes, or after 120 minutes, or after 240 minutes, or after 360 minutes, or after 480 minutes, or after 1440 minutes, when tested in a USP II apparatus using 900 ml of pH=6.8 buffer, 50 RPM, and 37° C.

A delayed release profile, where less than 5% of the API, or less than 10% of the API, is released from either the API granulates or the finished drug product formulated from the API granulates after one hour, when tested in a USP II apparatus using 900 ml of simulated gastric fluid with pH<2, 50 RPM, and 37° C.

The time period for 50% of the API to be released from either the API granulates or the finished drug product formulated from the API granulates is half, or less than half, of the time period required for 50% of the API to be released from a physical mix of the API and surfactant or a finished drug product formulated from a physical mix of the API and surfactant, respectively.

With respect to compactability, pharmaceutical dosage forms produced from the disclosed API granulates are highly compactible. For example, pharmaceutical dosage forms produced from the disclosed API granulates may be successfully formed into tablets by compression at a compaction pressure of 500 psi to 2000 psi, such as 600 psi to 1800 psi, or 800 psi to 1500 psi. In some embodiments, tablets produced by compression of API granulates as disclosed herein may be successfully compressed into tablets at a compaction pressure of 600, 800, 1000, or 1800 psi. In general, API granulates are successfully compressed into tablets if such tablets are durable and maintain their shape (e.g., cylindrical shape) during ordinary and API-appropriate handling, packaging, and storage prior to administration to a patient.

In some embodiments, the disclosed API granulates are capable of being compressed into tablets having an average diameter of about 5 mm to about 10 mm, such as about 5 mm, about 6 mm, about 7 mm, about 8 mm, about 9 mm, or about 10 nm.

In some embodiments, the disclosed API granulates are capable of being compressed into tablets having an average thickness of about 3 mm to about 6 mm, such as about 3 mm, about 4 mm, about 5 mm, or about 6 mm.

In some embodiments, the disclosed API granulates are capable of being compressed into tablets having an average breaking force (hardness) of about 50 N to about 125 N or more, such as about 55 N or more, about 60 N or more, about 75 N or more, or about 100 N or more. Testing of the breaking or crushing force of a tablet is well established in the art. Brittle tablets (those that break into two or more large pieces) are tested by pressing along one of their axes. Pressure is increased until the tablet cracks (brittle failure). The cracking event is characterized by a sudden decrease of the compression force, as the cracked tablet is no longer able to withstand the applied pressure. The force that causes the tablet to break is detected as the value of the compressive force immediately before the tablet cracks. Multiple commercial devices are readily available for the performance of this test. Fell & Newton, “Determination of Tablet Strength by the Diametral-Compression Test,” J. Pharm. Sci., 59 (5): 688-691 (1970).

With respect to flowability, the Carr index is an indicator of the flowability of a powder, and compares the difference between the bulk density and tapped density of a substance to determine its compressibility. Flowability as measured using the Carr Index may be determined in accordance with USP 1174. In some embodiments, the flowability of the disclosed API granules is about 16% to about 20%, such as about 17% to about 19% or about 18.1%±1.1%. Typically, powders/granules with Carr's Index of 16-20% are characterized as fair flowing powders (Shah et al., AAPS PharmSciTech, 9 (1): 250-258 (2008)).

### EXAMPLES

The disclosed technology is next described by means of the following non-limiting examples. The use of these and other examples anywhere in the specification is illustrative only, and in no way limits the scope and meaning of the invention or of any exemplified form. Likewise, the invention is not limited to any particular preferred embodiments described herein. Indeed, modifications and variations of the invention may be apparent to those skilled in the art upon reading this specification, and can be made without departing from its spirit and scope. The invention is therefore to be limited only by the terms of the claims, along with the full scope of equivalents to which the claims are entitled. Efforts have been made to ensure accuracy with respect to values presented (e.g., amounts, temperature, etc.), but some experimental error and deviation should be accounted for. Throughout the present disclosure, unless indicated otherwise, parts are parts by weight, molecular weight is weight average molecular weight, temperature is in degrees Centigrade and is at or near room temperature, pressure is at or near atmospheric, and, and a percentage content of an ingredient is a weight percentage.

### Example 1

This example describes the preparation and analysis of (i) an API (favipiravir) alone and (ii) favipiravir physically mixed with surfactant (poloxamer), as compared to (iii) favipiravir melt-coated and melt-granulated with poloxamer in accordance with the disclosed technology.

Favipiravir, also known as 6-fluoro-3-oxo-3,4-dihydropyrazine-2-carboxamide (molecular formula: C₅H₄FN₃O₂), has a melting point of 187° C. to 193° C. At a pH of 2.0-5.5, it is only slightly soluble. At a pH of 5.5-6.1, it is sparingly soluble. Its dissociation constant, pKa is 5.1. Favipiravir is difficult to formulate as it tends to easily form undesirable agglomerates and is poor flowing. Most particles of favipiravir have a size of less than 100 micrometers. Poloxamer generally functions as a dispersing agent, emulsifying agent, solubilizing agent, wetting agent, and lubricant. Many grades of Poloxamer have a melting point of 52° C. to 57° C.

The following samples were prepared:
Physical Mixture of favipiravir and 10 wt % Poloxamer 407 (P407) (melting point: 53-57° C.)
Physical Mixture of favipiravir and 10 wt % Poloxamer 188 (P188) (melting point: 55° C.)
Pure favipiravir
Melt-granulated and melt-coated (“treated”) blend of favipiravir and 10 wt % P188 at a processing temperature of 45° C.
Melt-granulated and melt-coated (“treated”) blend of favipiravir and 10 wt % P188 at a processing temperature of 55° C.

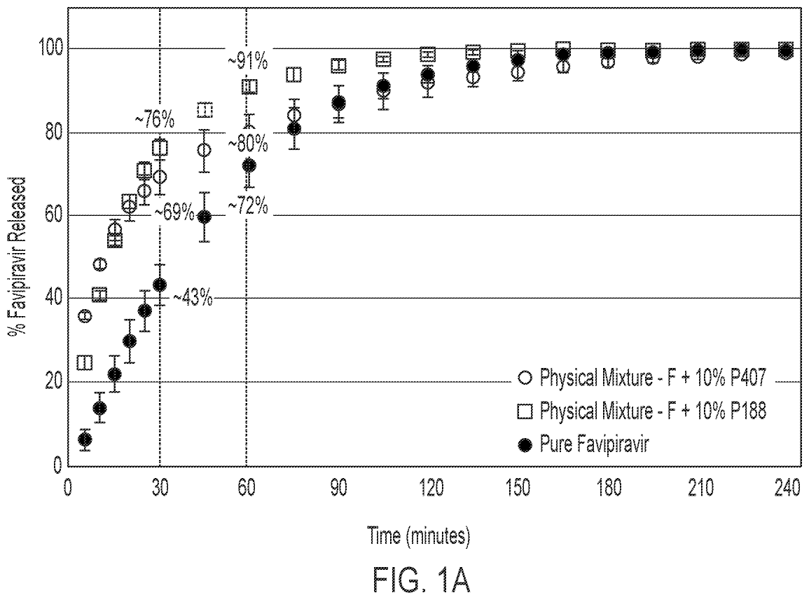

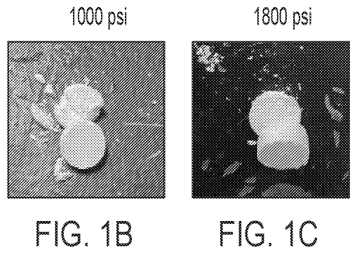

Each physical mixture was subjected to high shear mixing at room temperature using a LabRAM mixer at 70% intensity for 3 minutes. The poloxamers have dual functionality as both a surfactant and a binder. Dissolution testing was conducted on samples containing 800 mg of favipiravir. The samples were added to a dissolution vessel, and dissolution was conducted using the USP paddle method at 50 rpm in 900 ml of a medium including acetate buffer with a pH of 4.5. The results of the dissolution testing for samples 1, 2, and 3 are shown in FIG. 1A, which shows that both physical mixtures exhibited significant improvement in dissolution as compared to pure favipiravir. It was also observed that the P188-containing blend dissolves slightly faster. When sample 3 was subjected to a compaction pressure of 1000 psi, the results were unacceptable, crumbling, and non-cohesive. Similar unacceptable results were observed at 1800 psi compaction (FIGS. 1B-1C).

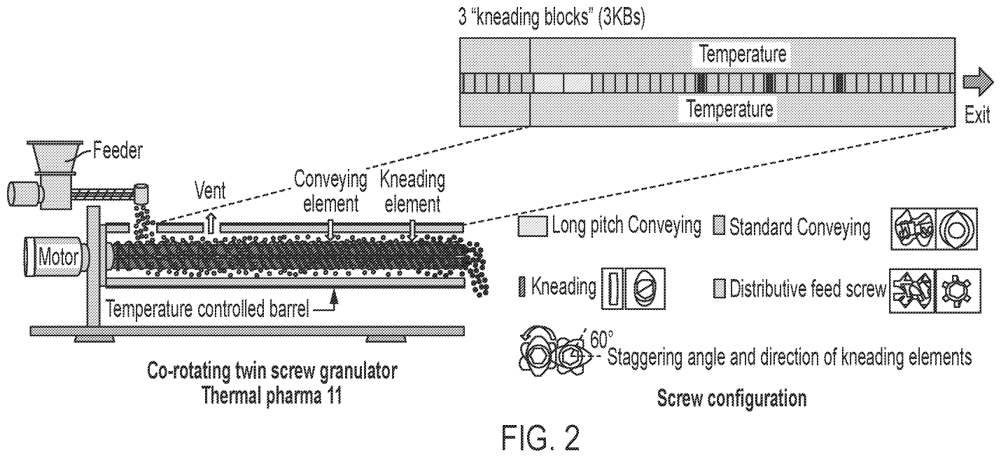

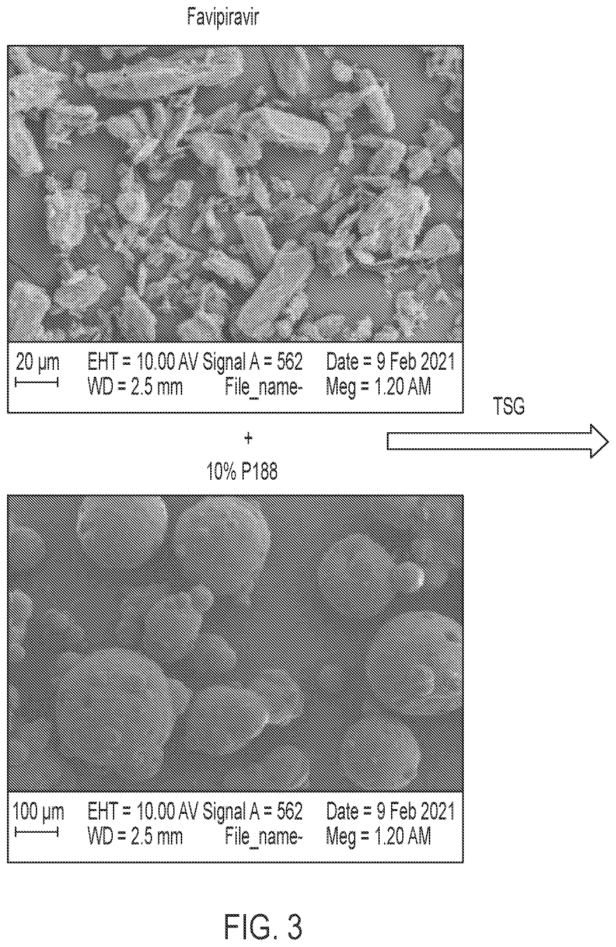

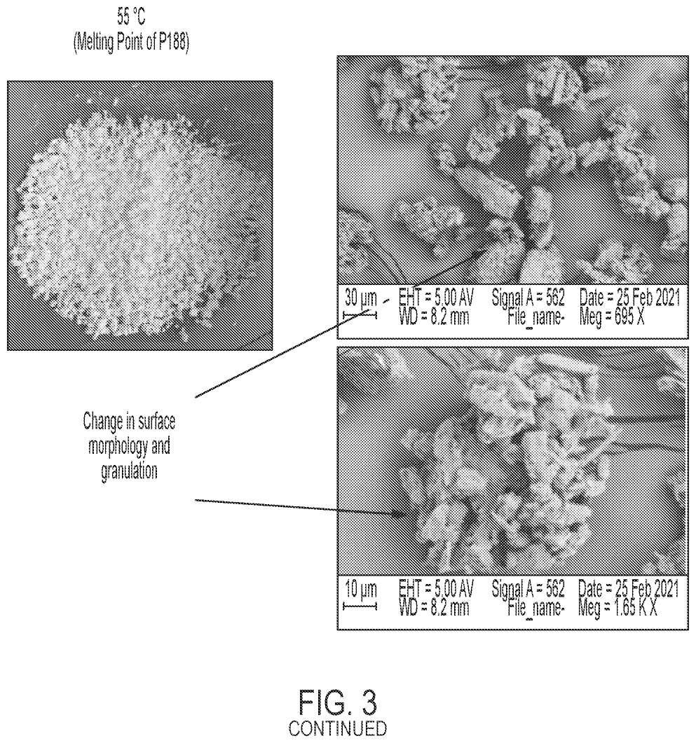

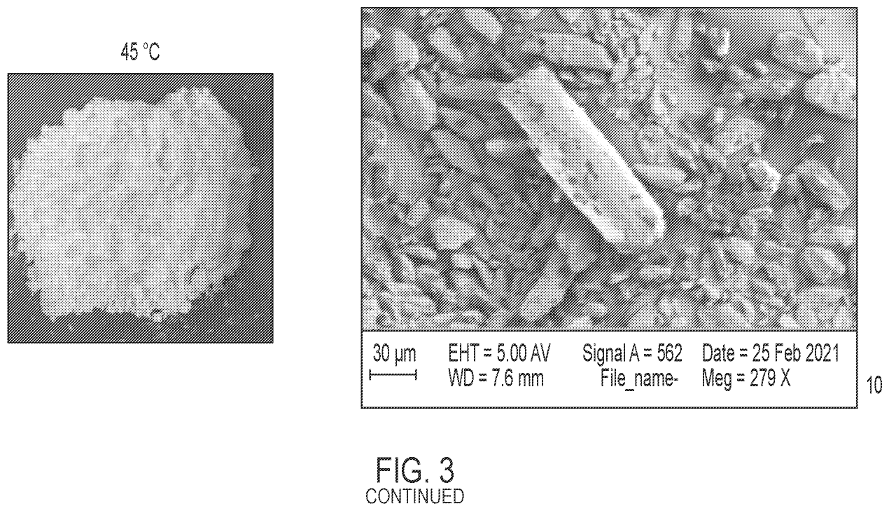

The melt-coated and melt-granulated blend of favipiravir and 10 wt % P188 was prepared by combining the favipiravir and P188 in a pre-mix using a LabRAM mixer and then feeding the blend into a co-rotating twin screw processor (Thermal pharma 11) at a throughput of 0.5 kg/hr, screw speed of 150 rpm, and at a processing temperature of either 45° C. or 55° C. The residence time of the blend in the twin screw processor was one minute or less. A schematic illustration of the processor is shown in FIG. 2. Using a processing temperature of 45° C. produced an API-containing powder but no API granulates. In contrast, with a processing temperature of 55° C., a change in surface morphology and granulation was observed, and API granulates were formed (FIG. 3).

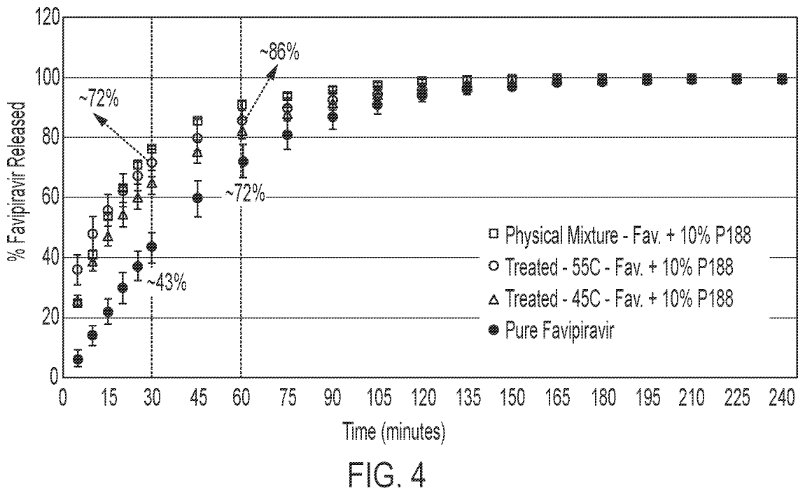

Additional dissolution testing was conducted samples 4 and 5 containing 800 mg of favipiravir. For this testing, the powder was added to a dissolution vessel, and dissolution was conducted using the USP paddle method at 50 rpm in 900 ml of a medium including acetate buffer with a pH of 4.5. The results of this dissolution testing are shown in FIG. 4 for samples 2, 3, 4, and 5. Notably, there was no improvement in dissolution rate for the treated blend that was processed at 45° C. when compared to the physical mixture; in fact, dissolution was slightly slower. However, it was surprisingly observed that granulates formed at a processing temperature of 55° C. (approximately equal to the melting point of the P188 surfactant) had a similar dissolution rate as the direct blend, and much faster than the pure favipiravir.

Particle (granule) size distribution of the treated blend of favipiravir and 10 wt % P188 processed at 55° C. was analyzed. Prior to co-milling, particle size distribution was characterized by D10 (584 micrometers), D50 (1196 micrometers); and D90 (1990 micrometers), as determined by laser light scattering or some other suitable method; and after co-milling, particle size distribution was characterized by D10 (350 micrometers), D50 (575 micrometers); and D90 (878 micrometers).

Compression of the treated blend of favipiravir and 10 wt % P188 processed at 55° C. was also analyzed. Specifically, co-milled granulates with no extra-granular addition were subjected to a Carver Manual Press for direct compression using 10 mm cylindrical flat tooling and 600 mg equivalent of favipiravir. The results of this analysis showed remarkably superior compactability as summarized in Table 2 and shown in FIG. 5. Even at a high compaction pressure of 1800 psi, the API granulates were successfully formed into tablets having excellent density, stability, durability, and hardness.

TABLE 2

| Compaction | Average | Average | Average |
|---|---|---|---|
| Pressure | Diameter | Thickness | Breaking |
| (psi) | (mm) | (mm) | Force (N) |
| 600 | 10.03 | 5.69 | 61.0 |
| 800 | 10.02 | 5.70 | 64.7 |
| 1000 | 10.03 | 5.66 | 67.3 |
| 1800 | 10.03 | 5.60 | 66.7 |

### Example 2

This example describes the preparation and analysis of immediate release API granulates including favipiravir, poloxamer, and intragranular addition of a disintegrant (CMC-Na). The following samples were prepared:
Treated blend of favipiravir+10 wt % P188, processing temp. 55° C.
Pure favipiravir
Treated blend of favipiravir+10 wt % P188+2% CMC-Na, processing temp. 55° C.
Treated blend of favipiravir+10 wt % P188+2% CMC-Na, processing temp. 60° C.

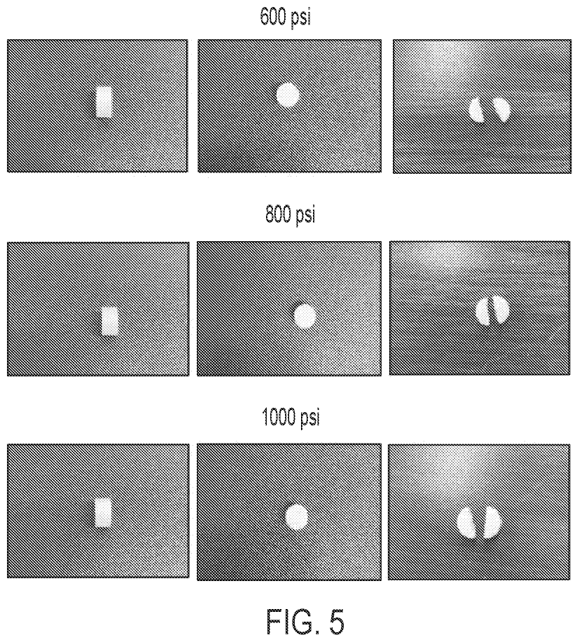

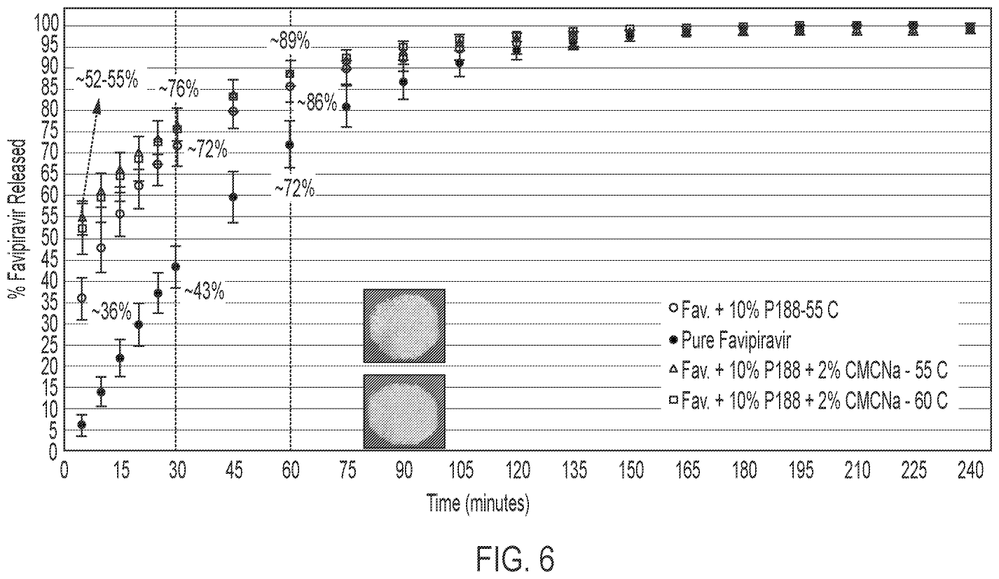

Dissolution testing was conducted on samples containing 800 mg favipiravir, which were added to a dissolution vessel, and dissolution was conducted using the USP paddle method at 50 rpm in 900 ml of a medium including acetate buffer with a pH of 4.5. Results from this dissolution testing of the above samples are shown in FIG. 6. It was observed that addition of disintegrant (CMC-Na) enabled a faster release, especially at the initial time points, as compared to API granulates prepared without disintegrant—i.e., very close to 90% release was obtained at 60 minutes.

### Example 3

This example describes the preparation and analysis of sustained release API tablets including favipiravir, poloxamer, and extra-granular addition of a sustained release agent, (HPMC-K15M Premium CR from Dow Chemicals; high molecular weight; good compaction binding properties) and a lubricant (MgSt). Prepared samples are listed in Table 3.

TABLE 3

| | Compaction | Average | Average | Average |
|---|---|---|---|---|
| | Pressure | Diameter | Thickness | Breaking |
| Granules | (psi) | (mm) | (mm) | Force (N) |
| [Fav. + 10% P188] + 10% | 1000 | 10.03 | 6.60 | 79.7 |
| HPMC K15M | 1800 | 10.04 | 6.55 | 84.7 |
| [Fav. + 10% P188 + 2% | 1800 | 10.02 | 6.67 | 106.5 |
| CMC-Na] + 10% | | | | |
| HPMC K15M | | | | |
| [Fav. + 5% P188 + 2% | 1800 | 10.06 | 6.10 | 60.5 |
| CMC-Na] + 15% | | | | |
| HPMC K15M | | | | |
| [Fav. + 10% P188 + 2% | 1800 | 10.02 | 6.64 | 120 |
| CMC-Na] + 10% HPMC | | | | |
| K15M + 0.5% MgSt | | | | |
| [Fav. + 10% P188 + 2% | 1800 | 10.02 | 6.2 | 116 |
| CMC-Na] + 5% HPMC | | | | |
| K15M + 0.5% MgSt | | | | |

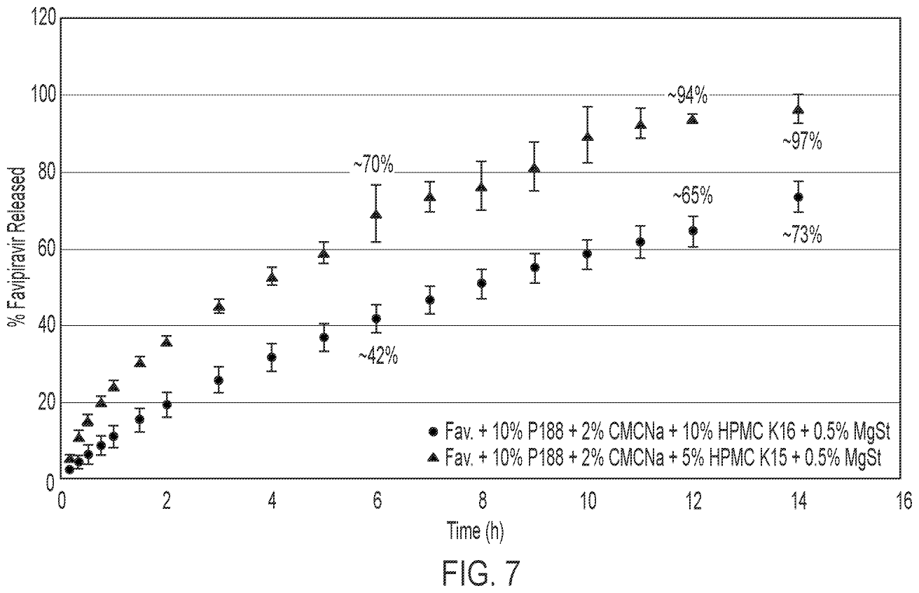

The presence of the meltable binder and disintegrant inside the API tablets led to superior compactability. Lubricant (MgSt) at 0.5% w/w and low shear acted as a compaction binder. Formulations listed in the last two rows of Table 3 were formed into tablets and then subjected to dissolution testing, the results of which are shown in FIG. 7. The data indicate that the release rate can be controlled by adding a small amount of HPMC; and that 5% w/w HPMC enabled the target release profile of 12 hours, based on a favipiravir dose recommendation of 600 or 800 mg.

### Example 4

This example describes the preparation and analysis of binder-free high-dose darunavir formulations manufactured by a process involving twin-screw melt granulation. An objective of this example is to evaluate the feasibility of a binder-free melt granulation process for high dose darunavir formulations while minimizing the amount of excipients, wherein the process provides an advantageous alternative to wet granulation processing. The evaluation considered the effect of process parameters on the granules (granule size distribution (GSD), porosity, bulk/tapped density, flowability) and on the properties (hardness and dissolution) of tablets made therefrom.

Darunavir (molecular formula: C₂₇H₃₇N₃O₇S), has a melting point of 74-76° C. and a solubility in water of 8.7 mg/L at 25° C. The structure of darunavir is shown below:

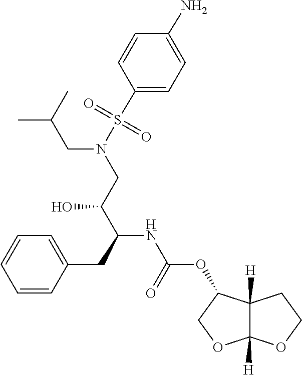

Experimental Set-up: A twin-screw processor was used having a screw configuration that included a powder inlet, three kneading zones, and distributive feed screws. Granules were released from the extruder.

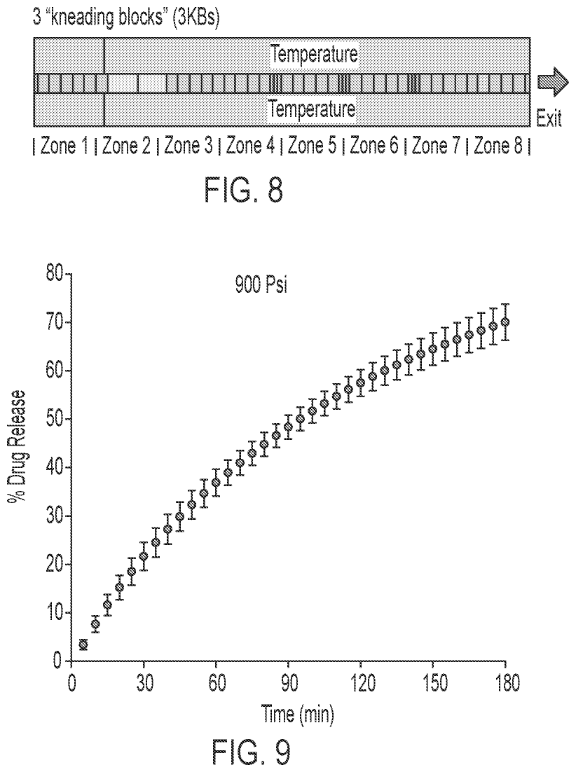

Initially, 23 runs were performed with different operating conditions and three heating zones (Zones 4-6, as shown in FIG. 8). All of the darunavir granules contained 100 wt % darunavir and were melt-granulated in the absence of any excipients. After granulation, an excipient (e.g., MgSt and/or CMC-Na) was incorporated extra-granularly. Successful granulation was possible with the use of three heating zones, but the process was not stable and insufficient control of the unheated zones resulted in material sticking. Utilizing gradual heating and seven heating zones led to a more stable granulation process. With better temperature control in all of the zones, melt granulation was possible even at 55° C., 20° C. lower than the melting point of darunavir. The temperature settings in the seven heating zones (Zones 2-8, as shown in FIG. 8) as provided in Table 4.

TABLE 4
SET POINTS

| | Throughput | Screw | T2 | T3 | T4 | T5 | T6 | T7 | T8 |
|---|---|---|---|---|---|---|---|---|---|
| Run # | (kg/h) | Speed (rpm) | (° C.) | (° C.) | (° C.) | (° C.) | (° C.) | (° C.) | (° C.) |
| 13 | 0.4 | 150 | 50 | 55 | 55 | 55 | 55 | 50 | 40 |
| 14 | 0.4 | 300 | 50 | 55 | 55 | 55 | 55 | 50 | 40 |
| 15 | 0.2 | 150 | 50 | 55 | 55 | 55 | 55 | 50 | 40 |
| 16 | 0.2 | 300 | 50 | 55 | 55 | 55 | 55 | 50 | 40 |
| 17 | 0.4 | 150 | 50 | 65 | 65 | 65 | 65 | 50 | 40 |
| 18 | 0.4 | 300 | 50 | 65 | 65 | 65 | 65 | 50 | 40 |
| 19 | 0.2 | 150 | 50 | 65 | 65 | 65 | 65 | 50 | 40 |
| 20 | 0.2 | 300 | 50 | 65 | 65 | 65 | 65 | 50 | 40 |

X-ray diffraction data showed that the crystalline structure of original darunavir particles did not change during the granulation process, regardless of whether three or seven zones were heated. Also, analysis of percent loss-on-drying (LOD) was also conducted with respect to Run 18 (processing temperature, 65° C.) comparing pure/ungranulated darunavir with granulated darunavir. The results of this LOD analysis are shown in Table 5, which indicates the percentage weight loss due to moisture evaporation.

TABLE 5

| | Pure Darunavir | Run 18 (65° C.) |
|---|---|---|
| Sample 1 | 4.36% | 3.49% |
| Sample 2 | 4.28% | 3.64% |
| Sample 3 | 4.63% | 3.29% |
| Avg. | 4.42% | 3.47% |

Granulation size distribution (GSD) results from Runs 13-20 are shown in Table 6.

TABLE 6

| | GSD | | |
|---|---|---|---|
| Run | D10 (um) | D50 (um) | D90 (um) |
| 13 | 313 | 1011 | 2164 |
| 14 | 438 | 1140 | 2220 |
| 15 | 414 | 1045 | 2103 |
| 16 | 568 | 1175 | 2141 |
| 17 | 400 | 1173 | 2274 |
| 18 | 432 | 1319 | 2349 |
| 19 | 533 | 1260 | 2195 |
| 20 | 547 | 1231 | 2256 |

The following formulations were utilized to prepare tablets from darunavir granules using 10 mm or 12 mm flat cylindrical tooling:
99% Darunavir/Granules+1% MgSt
96% Darunavir/Granules+1% MgSt+3% CMC-Na
91% Darunavir/Granules+1% MgSt+3% CMC-Na+5% Ceolus/Prosolv

This provided an assessment of granule tabletability using a PRESSTER compactor simulator, producing 12 mm flat cylindrical tablets having a thickness of approximately 4.6 mm. The results of this assessment are provided in Table 7. As shown below, a small amount of excipients can produce tablets with high hardness for standard diameters.

TABLE 7

| | | | | Avg. Pre- | Avg. Main | Avg. | Tablet | Avg. Crushing | |
|---|---|---|---|---|---|---|---|---|---|
| Tablet | | Darunavir | Total Tablet | Compression | Compression | Ejection | density | Force/ | Tensile |
| Batch | Formulation | Content (mg) | Weight (mg) | Force (kN) | Force (kN) | Force (N) | (g/cm³) | Hardness (N) | Strength (MPa) |
| Pure | 99% darunavir | 600 | 606 | 1.80 | 13.26 | 585.84 | 1.14 | 42.9 | 0.49 |
| Darunavir | powder + 1% MgSt | | | | | | | | |
| Pure | 99% darunavir | 600 | 606 | 9.59 | 14.45 | 603.01 | 1.15 | 36.8 | 0.42 |
| Darunavir | powder + 1% MgSt | | | | | | | | |
| Run 13 | 99% darunavir | 600 | 606 | 1.67 | 14.70 | 603.01 | 1.14 | 58.6 | 0.67 |
| | granules + 1% MgSt | | | | | | | | |
| Run 14 | 99% darunavir | 600 | 606 | 9.94 | 14.8 | 455.38 | 1.15 | 77.2 | 0.88 |
| | granules + 1% MgSt | | | | | | | | |
| Run 16 | 99% darunavir | 600 | 606 | 9.95 | 14.72 | 413.38 | 1.15 | 69.6 | 0.80 |
| | granules + 1% MgSt | | | | | | | | |
| Run 21 | 99% darunavir | 600 | 606 | 9.9 | 15.03 | 429.95 | 1.15 | 72.0 | 0.82 |
| | granules + 1% MgSt | | | | | | | | |
| Run 23 | 99% darunavir | 600 | 606 | 10.16 | 15.15 | 491.46 | 1.15 | 77.2 | 0.88 |
| | granules + 1% MgSt | | | | | | | | |
| Pure | 96% darunavir | 600 | 625 | 1.72 | 12.82 | 537.86 | 1.16 | 65.3 | 0.76 |
| Darunavir | powder + 1% MgSt + | | | | | | | | |
| | 3% CMC—Na | | | | | | | | |
| Run 17 | 96% darunavir | 600 | 625 | 1.84 | 14.30 | 578.83 | 1.15 | 95.2 | 1.09 |
| | granules + 1% MgSt + | | | | | | | | |
| | 3% CMC—Na | | | | | | | | |
| Run 19 | 96% darunavir | 600 | 625 | 1.54 | 13.84 | 539.41 | 1.15 | 75.5 | 0.87 |
| | granules + 1% MgSt + | | | | | | | | |
| | 3% CMC—Na | | | | | | | | |

Granules of pure darunavir were prepared and sieved to obtain three different grades of granules (Table 8). The processing parameters for the granules in table 8 were: 0.4 kg/h throughput, 300 rpm screw speed, 60° C. processing temperature.

TABLE 8

| Granule | Pre- | Main | Ejection | | Avg. Crushing |
|---|---|---|---|---|---|
| size | Compression | Compression | Force | Weight | Force/ |
| (μm) | Force (kN) | Force (kN) | (N) | (mg) | Hardness (N) |
| −850 | 7.0 | 13.2 | 484.4 | 303 | 55 |
| 850-1400 | 7.0 | 14.3 | 486.4 | 303 | 35 |
| 1400+ | 6.6 | 12.8 | 450.4 | 303 | 33 |

The granules were blended with 1% of MgSt and formed into tablets using 10 mm tooling. Desirably hard tablets were only obtained with granule size under 850 μm (hardness=55N). Layering and capping issues were observed with granule size above 850 μm, and the tablet hardness was lower.

Particle Size Distribution (PSD) After Co-Milling: The granules of pure darunavir prepared as described above were passed through a Comil with a screen size of 1016 microns. PSD was determined using a light scattering method. The D50 range of the milled granules was 326 μm to 436 μm. Some of the runs has D90 larger than 1016 μm (Table 9), possibly because the granules may be elongated, or due to overlapping particles in the sizer.

TABLE 9

| | Run 13 | Run 17 | Run 18 | Run 19 | Run 20 |
|---|---|---|---|---|---|
| D10 (μm) | 17 | 23 | 21 | 22 | 27 |
| D50 (μm) | 376 | 436 | 326 | 330 | 415 |
| D90 (μm) | 887 | 915 | 1137 | 1070 | 1046 |

Granule Tabletability—Manual Press: Tablets were prepared and analyzed using milled granules of darunavir and CEOLUS (microscrystalline cellulose) and 10 mm tooling. The tablets contained: 5 wt % CEOLUS, 3 wt % CMC-Na, 1 wt % MgSt, and 91 wt % darunavir. The highest average hardness was 144 N (Table 10).

TABLE 10

| | | Tablet | Compression | Tablet | Avg. Crushing |
|---|---|---|---|---|---|
| Batch | Tooling | weight | pressure | thickness | Force/ |
| number | (mm) | (mg) | (PSI) | (mm) | Hardness (N) |
| 1 | 10 | 660 | 500 | 7.1 | 87 |
| 2 | 10 | 660 | 700 | 6.9 | 108 |
| 3 | 10 | 660 | 900 | 6.8 | 144 |

### Example 5

This example relates to dissolution testing of darunavir-containing granules and tablets as disclosed herein.

A dissolution test was performed on darunavir-containing tablets using a USP Apparatus 2, speed 75 RPM, volume 900 ml, medium 2% tween 20 in 0.05 phosphate buffer pH 3, temperature 37° C. Tablet formulation: 91 wt % pure darunavir granules; 5 wt % CEOLUS, 3 wt % CMC-Na, 1 wt % MgSt. The dissolution results are shown in FIG. 9.

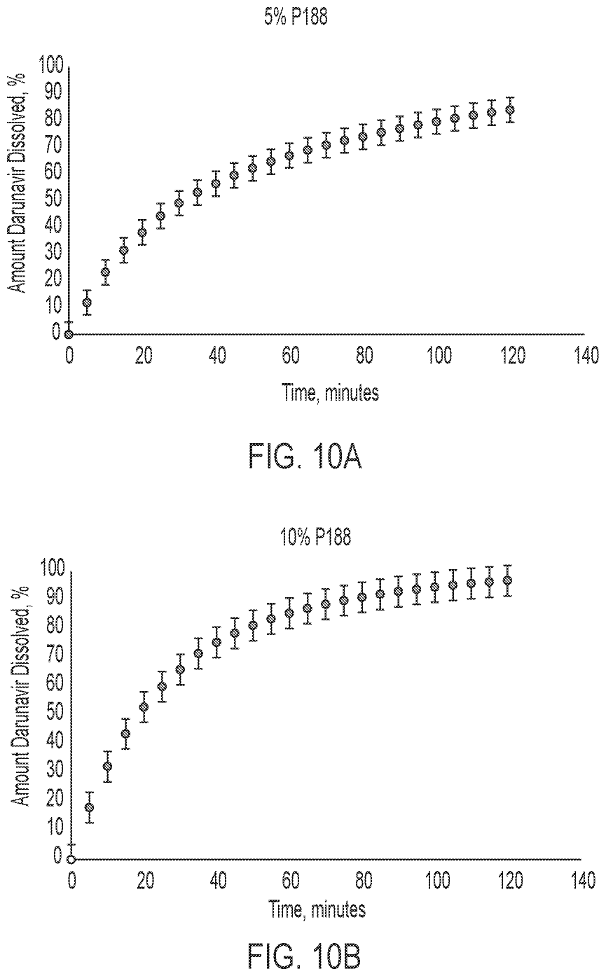

Additional dissolution tests were performed on darunavir-containing granules to demonstrate the effect of melt-granulation (barrel temperature: 45° C.) with a surfactant/binder, Poloxamer P188. Dissolution conditions: USP Apparatus 2; RPM: 75; Volume: 900 ml; Medium: 2% Tween 20 in 0.05 Phosphate Buffer (pH 3); Temp: 37° C. The following intragranular formulations were prepared and tested:
Intragranular Formulation: 91.5 wt % Darunavir, 5 wt % P188, 3 wt % Crospovidone. Result: ˜67% Darunavir released in 1 hour (FIG. 10A).
Intragranular Formulation: 86.5 wt % Darunavir, 10 wt % P188, 3 wt % Crospovidone. Result: ˜86% Darunavir released in 1 hour (FIG. 10B).

Additional dissolution tests were performed on darunavir-containing tablets to demonstrate the effect of adding intra/extra granular disintegrant (crospovidone). Dissolution conditions: USP Apparatus 2; RPM: 75; Volume: 900 ml; Medium: 2% Tween 20 in 0.05 Phosphate Buffer (pH 3); Temp: 37° C. All of the tablets were made by manual press under the same compression force. The following tablet formulations were prepared and tested:
Intra-granular Formulations: (Balance) Darunavir, 10 wt % P188, Crospovidone (0 and 3 wt %)
Extra-granular Formulations: Crospovidone (0 wt %, 2 wt %, and 5 wt %), 0.5 wt % MgSt, Darunavir (Balance)

Effect of extra-granular disintegrant addition. A darunavir tablet with no extra-granular disintegrant exhibited poor dissolution (5% in 1 hour), and a tablet hardness of 145N. A darunavir tablet with 2 wt % extra-granular disintegrant exhibited poor dissolution (30% in 1 hour) and a tablet hardness of 149N.

Effect of extra-granular and intra-granular disintegrant addition. A darunavir tablet with 3 wt % intra-granular disintegrant and 5 wt % extra-granular disintegrant exhibited a desirable immediate release dissolution (80% in 1 hour) and a tablet hardness of 211N. Hence, the inclusion of both intra- and extra-granular disintegrant corresponded to improved immediate release.

Effect of extra-granular disintegrant addition. A darunavir tablet with no intra-granular disintegrant and 5 wt % extra-granular disintegrant exhibited poor dissolution (40% in 1 hour) and a tablet hardness of 191N.

### Example 6

This example relates to analysis of the effect of pre-blending intensity and duration of exposure to heat and shear forces in a LabRAM mixer. Dissolution conditions: USP Apparatus 2; RPM: 75; Volume: 900 ml; Medium: 2% Tween 20 in 0.05 Phosphate Buffer (pH 3); Temp: 37° C. The results are shown in Table 11.

TABLE 11

| | LabRam | | | |
|---|---|---|---|---|
| Intra-granular | intensity/ | Initial | | |
| Formulation | duration | product | Subsequent processing | Dissolution profile |
| 89.4 wt % darunavir; | 40% for 1 | White | Granules obtained by | Tablets: 40% darunavir |
| 10.6 wt % P188 | min | powder | TSG, then 5% | released in 1 hr |
| | | | Crospovidone, 0.5% MgSt | |
| | | | added to make tablets | |
| 89.4 wt % darunavir; | 70% for 5 | White | Granules obtained by | Granules: 58% darunavir |
| 10.6 wt % P188 | min | powder | TSG, then 5% | released in 1 hr |
| | | | Crospovidone, 0.5% MgSt | Tablets: 26% darunavir |
| | | | added to make tablets | released in 1 hr |
| 89.4 wt % darunavir; | 80% for 10 | Granules | Tablets made by adding | Tablets: 81% darunavir |
| 10.6 wt % P188 | min | | 5% crospovidone and | released in 1 hr |
| | | | 0.5% MgSt | |
| 86.2 wt % darunavir; | 70% for 5 | White | Granules obtained by | Tablets: 60% darunavir |
| 10.6 wt % P188; 3.2 | min | powder | TSG, then 5% | released in 1 hr |
| wt % crospovidone | | | Crospovidone, 0.5% MgSt | |
| | | | added to make tablets | |

### Example 7

This example relates to analysis of the effect of mixing multiple excipients in step (a) to vary the composition of the melt-coated/melt-granulated granules. A blend was prepared in the lab ram containing 80 wt % darunavir, 10 wt % P188, 3 wt % low molecular weight HPMC (a wetting agent), 2 wt % high molecular weight HPMC (a controlled release agent), and 5 wt % lactose (a filler). The mixture was melt-coated/melt-granulated. The resulting granules were then milled in a Comil with a screen size of 1016 microns. The resulting granules were mixed with 1 wt % MgSt, and then compacted into tablets containing 600 mg of darunavir using a 10 mm flat punch at a compaction pressure of 300 psi, to produce tablets with a crushing hardness of 162 N.

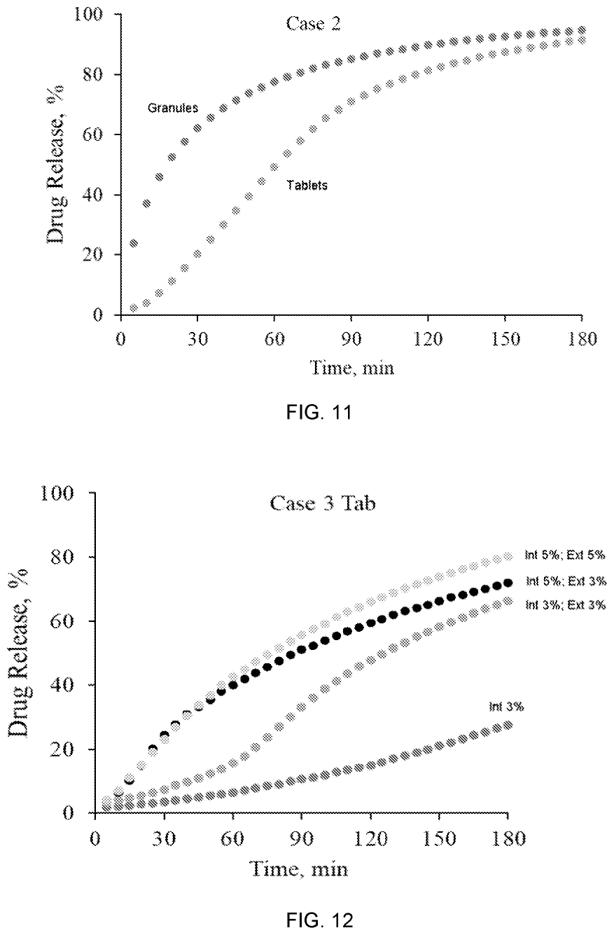

Samples of the granules and tablets containing 600 mg of darunavir were tested in a USP Apparatus 2; RPM: 75; Volume: 900 ml; Medium: 2% Tween 20 in 0.05 Phosphate Buffer (pH 3); Temp: 37° C. The results are shown in FIG. 11. While the granules dissolve relatively fast, as was also the case of Favipiravir in Example 3, even a small amount of HPMC is able to slow down dissolution significantly.

### Example 8

This example relates to analysis of the effect of mixing multiple excipients in steps (a) and (c) to vary the composition of the dosage form.

Blends was prepared in the lab ram containing darunavir, P188, and various amounts of NaCMC. The mixture was melt-coated/melt-granulated. The resulting granules were then milled in a Comil with a screen size of 1016 microns. The milled granules were mixed with low molecular weight HPMC, high molecular weight HPMC, lactose, MgSt and various amounts of NaCMC, to obtain final blends with the compositions shown in Table 12.

TABLE 12

| Composition of | | | Composition of tablet (wt %) | | | | | |
|---|---|---|---|---|---|---|---|---|
| granules (wt %) | | | | LMW | HMW | | | |
| Darunavir | P188 | Crospovidone | Granules | HPMC | HPMC | Lactose | Crospovidone | MgSt |
| 87 | 10 | 3 | 92 | 2 | 2 | 3.5 | 0 | 0.5 |
| 87 | 10 | 3 | 89 | 2 | 2 | 3.5 | 3 | 0.5 |
| 85 | 10 | 5 | 89 | 2 | 2 | 3.5 | 3 | 0.5 |
| 85 | 10 | 5 | 87 | 2 | 2 | 3.5 | 5 | 0.5 |

The blends were then compacted into tablets containing 600 mg of darunavir using a 10 mm flat punch at a compaction pressure of 300 psi. The API concentration in these tablets ranged from about 74 wt % to about 80 wt %.

Dissolution of these tablets was tested in a USP Apparatus 2; RPM: 75; Volume: 900 ml; Medium: 2% Tween 20 in 0.05 Phosphate Buffer (pH 3); Temp: 37° C. The results are shown in FIG. 12, demonstrating that a very wide range of dissolution profiles can be obtained using the melt-coated/melt-granulated granules in combination with small amounts of intra- and extra-granular excipients.

### Example 9

This example relates to analysis of the effect of including at least one additional API in a dosage form that contains melt-coated/melt-granulated darunavir.

Darunavir, Chlorpheniramine Maleate (CPM, an antihistamine), Ibuprofen (Ibu, an analgesic) and Dexamethasone (Dexa, a steroidal anti-inflammatory drug) were selected to form granules with different compositions, as shown in Table 13.

TABLE 13

| | Composition of granules (wt %) | | | | | | Composition of tablet (wt %) | | | | | |
|---|---|---|---|---|---|---|---|---|---|---|---|---|
| | | | Cross- | | | | | Cross- | | | | |
| Sample | Darunavir | P188 | povidone | CPM | Ibu | Dexa | Granules | povidone | CPM | Ibu | Dexa | MgSt |
| 1 | 69.6 | 10 | 3 | 17.4 | 0 | 0 | 99.5 | 0 | 0 | 0 | 0 | 0.5 |
| 2 | 69.6 | 10 | 3 | 0 | 17.4 | 0 | 99.5 | 0 | 0 | 0 | 0 | 0.5 |
| 3 | 69.6 | 10 | 3 | 0 | 0 | 17.4 | 99.5 | 0 | 0 | 0 | 0 | 0.5 |
| 4 | 87 | 10 | 3 | 0 | 0 | 0 | 79.5 | 3 | 17 | 0 | 0 | 0.5 |
| 5 | 87 | 10 | 3 | 0 | 0 | 0 | 79.5 | 3 | 0 | 17 | 0 | 0.5 |
| 6 | 87 | 10 | 3 | 0 | 0 | 0 | 79.5 | 3 | 0 | 0 | 17 | 0.5 |

In all cases, the mixture obtained after step (a) was melt-coated/melt-granulated and then milled using a Comil with a screen size of 1016 microns. The granules were then mixed with extra-granular ingredients as per the compositions in Table 13. The final mixtures were then compressed into tablets using flat 10 mm punches and a compaction pressure of 300 psi.

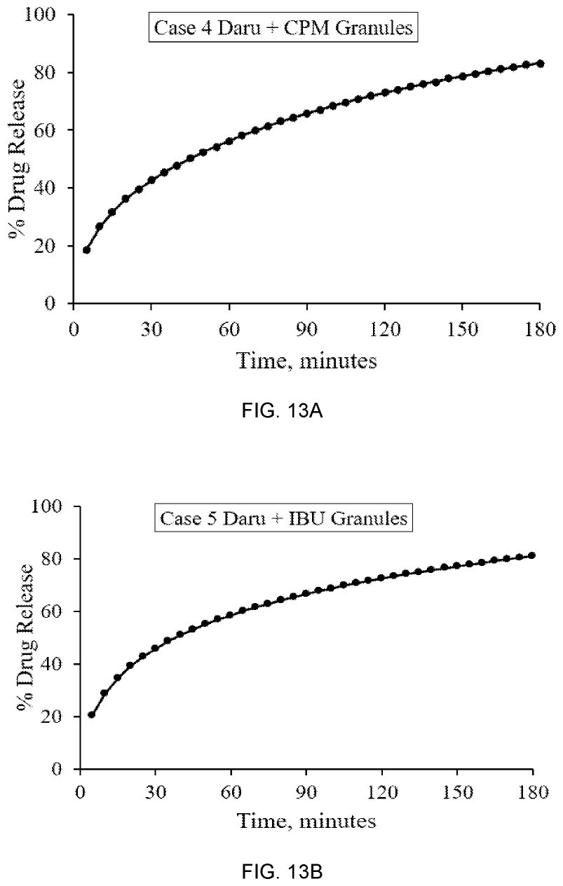

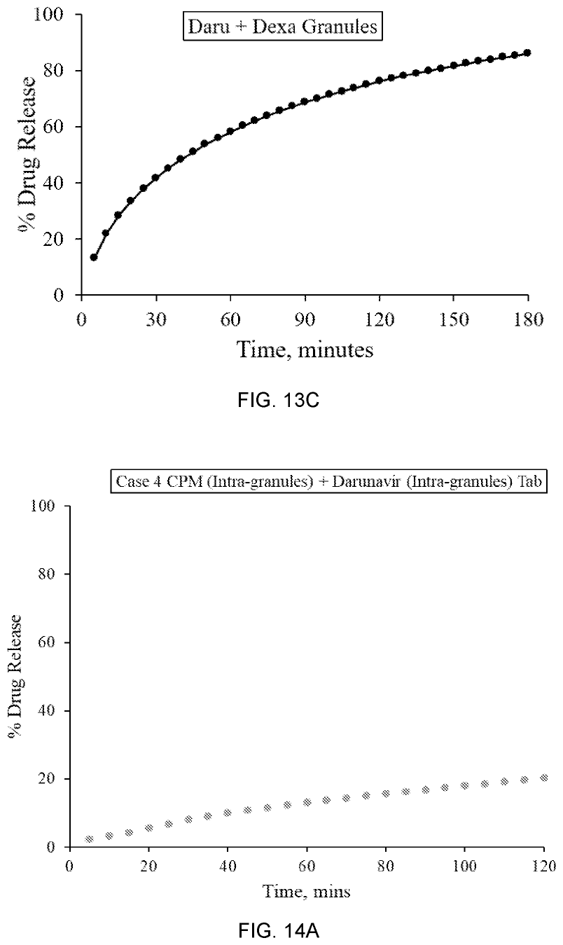

FIGS. 13A, 13B, and 13C, respectively, show the dissolution profiles obtained when the granules from samples 1, 2, and 3 were tested for dissolution using a USP Apparatus 2; RPM: 75; Volume: 900 ml; Medium: 2% Tween 20 in 0.05 Phosphate Buffer (pH 3); Temp: 37° C. In all three cases, the granules dissolved slightly more slowly than the granules containing only darunavir, 10% P188 and 3% cross-povidone (FIG. 10B). This indicates that the second API is likely competing with darunavir for the surfactant.

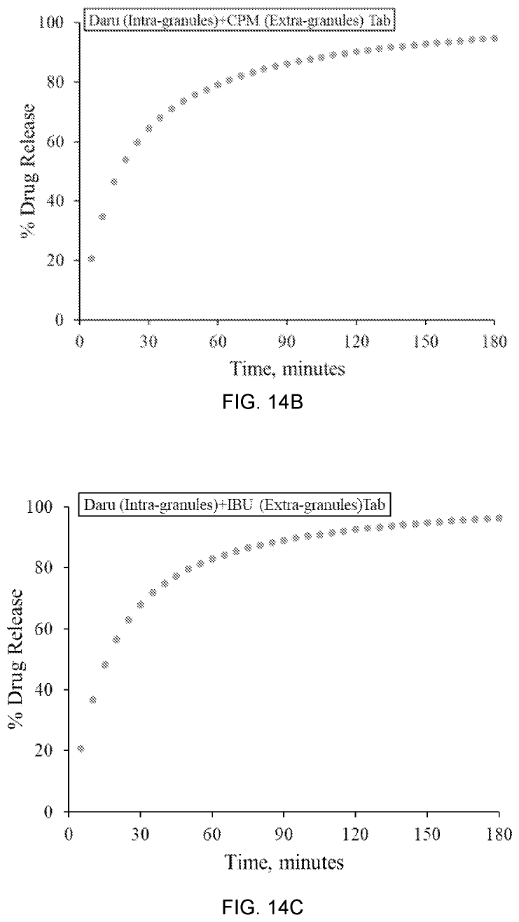

The tablets obtained in sample 1 were also tested for dissolution using a USP Apparatus 2; RPM: 75; Volume: 900 ml; Medium: 2% Tween 20 in 0.05 Phosphate Buffer (pH 3); Temp: 37° C. As shown in FIG. 14A, these tablets showed much slower dissolution than the granules. This result is surprising because in this case the second API, CPM, is readily soluble in the dissolution medium. Very slow dissolution was also observed for tablets corresponding to samples 2 and 3.

However, as shown in FIG. 14B, when the only API inside the granules was darunavir, and the second API was added in step (c) (i.e., as an extra-granular ingredient) dissolution of the tablets corresponding to samples 4, 5, and 6 exhibited substantial enhancement. This surprising result clearly indicates that when multiple APIs are inside the granules, they compete for the surfactant, and the enhancement of dissolution is reduced. However, when the only API inside the granules is the poorly soluble darunavir, the full benefit of melt-coating is realized. The presence of the second API outside the granules does not hinder dissolution of darunavir. Similar results were observed for sample 6, where Dexamethasone was placed outside the darunavir granules.

The foregoing merely illustrates the principles of the disclosure. Any examples set forth in this specification are not intended to be limiting and merely set forth some of the many possible embodiments for the appended claims. Those skilled in the art will readily recognize various modifications and changes that may be made without following the example embodiments and applications illustrated and described herein, and without departing from the true spirit and scope of the following claims.

All references cited and/or discussed in this specification are incorporated herein by reference in their entireties and to the same extent as if each reference was individually incorporated by reference.

We claim:

1. A process for manufacturing an oral pharmaceutical dosage form comprising:
(a) mixing a poorly soluble active pharmaceutical ingredient (API) and a surfactant into a blend;
(b) feeding the blend into a processor that applies heat and shear forces to the blend at a processing temperature within a range of approximately the melting point of the surfactant to 3° C. below the melting point of the surfactant so as to form melt-coated, melt-granulated API granulates; and
(c) formulating the API granulates into a sustained release oral pharmaceutical dosage form;
wherein the API content in the dosage form is at least 60%, at least 70%, at least 80%, or at least 90% by weight, based on the total weight of the dosage form.

2. The process of claim 1, wherein the processing temperature is ranges from the melting point of the surfactant to 2° C. below the melting point of the surfactant.

3. The process of claim 1 or 2, wherein the processing temperature is approximately equal to the melting point of the surfactant.

4. The process of any one of claims 1-3, wherein the API is poorly soluble.

5. The process of any one of claims 1-4, wherein the API is an antibiotic, an anti-parasitic agent, an antiviral, an analgesic, an anti-cancer agent, an anti-inflamatory agent, or any other API that requires a dosing above 300 mg per unit dose.

6. The process of any one of claims 1-5, wherein the API is selected from favipiravir, ibuprofen, carbamazepine, fenofibrate, indomethacin, imatinib, flufenamic acid, erlotinib hydrochloride, vitamin D, estradiol, and combinations thereof.

7. The process of any one of claims 1-6, wherein step (a) further comprises mixing at least one additional API into the blend.

8. The process of claim 7, wherein the at least one additional API is selected from a steroid, an anti-inflammatory agent, a non-steroidal anti-inflammatory drug, an antibiotic, an antiviral agent, an anti-cancer agent, an analgesic, an anti-histaminic agent, and combinations thereof.

9. The process of any one of claims 1-8, wherein the surfactant comprises one or more of poloxamer, polyoxyethylene stearate, cetylpyridinium chloride, polysorbate, and glyceryl monostearate.

10. The process of any one of claims 1-9, wherein step (a) further comprises mixing into the blend one or more pharmaceutically acceptable excipients selected from controlled release agents, carriers, fillers, extenders, binders, humectants, disintegrants, absorption accelerators, wetting agents, absorbents, lubricants, coloring agents, and diluents.

11. The process of any one of claims 1-10, wherein step (c) comprises combining the API granulates with one or more pharmaceutically acceptable excipients selected from controlled release agents, carriers, fillers, extenders, binders, humectants, disintegrants, absorption accelerators, wetting agents, absorbents, lubricants, coloring agents, and diluents.

12. The process of claim 10 or 11, wherein the one or more pharmaceutically acceptable excipients comprises a controlled release agent.

13. The process of claim 12, wherein the one or more pharmaceutically acceptable excipients comprises one or more of hydroxypropyl methylcellulose, crospovidone, sodium carboxymethyl cellulose, and methyl cellulose.

14. The process of any one of claims 1-13, wherein the dosage form is selected from a tablet, a capsule, and a powder.

15. The process of any one of claims 1-14, wherein step (c) comprises subjecting the API granulates to a compaction pressure to form a tablet, wherein the compaction pressure is greater than or equal to a pressure selected from 300 psi, 400 psi, 500 psi, 600 psi, 1000 psi, and 1800 psi.

16. The process of any one of claims 1-15, wherein: (i) steps (a) and (b) are performed as part of a continuous process, (ii) steps (b) and (c) are performed as part of a continuous process, or (iii) steps (a), (b), and (c) are performed as part of a continuous process.

17. The process of any one of claims 1-16, wherein the process is operated under closed loop control using a combination of sensors, controllers, and actuators to maintain the process within a desired range of operating parameters.

18. A dosage form prepared by the process of any one of claims 1-17.

19. An oral pharmaceutical dosage form comprising granulates of a poorly soluble API, wherein the dosage form has a total API content of at least 60 wt %, at least 70 wt %, at least 80 wt %, or at least 90 wt % and a surfactant content of less than or equal to 10 wt %, based on the total weight of the dosage form; and wherein the API granulates are capable of being compressed into tablets at a compaction pressure greater than or equal to a pressure selected from 300 psi, 400 psi, 500 psi, 600 psi, 1000 psi, and 1800 psi.

20. The dosage form of claim 19, wherein the dosage form is selected from a tablet, a capsule, and a powder.

21. The dosage form of any one of claim 19 or 20, wherein the dosage form is a tablet having an average breaking force of more than 50 N.

22. The dosage form of any one of claims 18-21, wherein the dosage form has faster dissolution than a comparative dosage form that differs only by having been made from a physical mix of the API and surfactant, as determined by the time required to release 80% of the darunavir from the dosage form, when tested using any one of the following:
(1) a USP II apparatus, 900 ml vessel, 75 RPM, using deionized water;
(2) a USP II apparatus, 900 ml vessel, 75 RPM, using pH 2 simulated gastric fluid; or
(3) a USP II apparatus, 900 ml vessel, 75 RPM, using pH 6.8 buffer.

23. A sustained release oral pharmaceutical dosage form comprising pure API granulates, wherein the dosage form: (i) has a total API content of at least 95 wt % based on the total weight of the dosage form; (ii) comprises about 200 mg to about 800 mg total API; and (iii) has an average breaking force of at least 50 N.

24. A process for manufacturing an immediate release darunavir-containing oral pharmaceutical dosage form, comprising:
(a) mixing darunavir and a surfactant into a blend;
(b) feeding the blend into a processor that applies heat and shear forces to the blend at a processing temperature within a range of approximately the melting point of the surfactant to 3° C. below the melting point of the surfactant so as to form melt-coated, melt-granulated granulates; and
(c) formulating the granulates into an immediate release oral pharmaceutical dosage form.

25. The process of claim 24, wherein the processing temperature ranges from approximately the melting point of the surfactant to 2° C. below the melting point of the surfactant.

26. The process of claim 24 or 25 wherein the processing temperature is approximately equal to the melting point of the surfactant.

27. The process of any one of claims 24-26, wherein the surfactant comprises one or more of poloxamer, polyoxyethylene stearate, cetylpyridinium chloride, polysorbate, and glyceryl monostearate.

28. The process of any one of claims 24-27, wherein step (a) further comprises mixing into the blend at least one additional active pharmaceutical ingredient (API) other than darunavir.

29. The process of any one of claims 24-27, wherein step (c) further comprises mixing the granules with at least one additional active pharmaceutical ingredient (API) other than darunavir.

30. The process of claim 29, wherein the at least one additional API is selected from a steroid, an anti-inflammatory agent, a non-steroidal anti-inflammatory drug, an antibiotic, an antiviral agent, an anti-cancer agent, an analgesic, an anti-histaminic agent, and combinations thereof.

31. The process of any one of claims 24-30, wherein step (a) further comprises mixing into the blend one or more pharmaceutically acceptable excipients selected from controlled release agents, carriers, fillers, extenders, binders, humectants, disintegrants, absorption accelerators, wetting agents, absorbents, lubricants, coloring agents, and diluents.

32. The process of any one of claims 24-31, wherein step (c) further comprises combining the granulates with one or more pharmaceutically acceptable excipients selected from controlled release agents, carriers, fillers, extenders, binders, humectants, disintegrants, absorption accelerators, wetting agents, absorbents, lubricants, coloring agents, and diluents.

33. The process of claim 31 or 32, wherein the one or more pharmaceutically acceptable excipients comprises a disintegrant.

34. The process of any one of claims 24-33, wherein the dosage form is selected from a tablet, a capsule, and a powder.

35. The process of any one of claims 24-34, wherein step (c) comprises subjecting the granulates to compression at a compaction pressure of greater than or equal to 600 psi to form a tablet.

36. The process of any one of claims 24-35, wherein: (i) steps (a) and (b) are performed as part of a continuous process, (ii) steps (b) and (c) are performed as part of a continuous process, or (iii) steps (a), (b), and (c) are performed as part of a continuous process.

37. The process of any one of claims 24-36, wherein the process is operated under closed loop control using a combination of sensors, controllers, and actuators to maintain the process within a desired range of operating parameters.

38. An immediate release darunavir-containing oral pharmaceutical dosage form prepared by the process of any one of claims 24-37.

39. An immediate release darunavir-containing oral pharmaceutical dosage form comprising granulates comprising darunavir and surfactant, wherein the dosage form has a darunavir content of at least 75 wt % and a surfactant content of less than or equal to 10 wt %, based on the total weight of the dosage form; and wherein the granulates are capable of being compressed into tablets at a compaction pressure of greater than 300 psi, greater than 400 psi, greater than 500 psi, greater than 600 psi, greater than 1000 psi, or greater than 1800 psi.

40. The dosage form of claim 38 or 39, wherein the granulates further comprise at least one additional active pharmaceutical ingredient other than darunavir.

41. The dosage form of claim 38 or 39, wherein the at least one additional active pharmaceutical ingredient other than darunavir is added in step (c) as an extragranular ingredient.

42. The dosage form of any one of claims 38-41, wherein the dosage form is selected from a tablet, a capsule, and a powder.

43. The dosage form of any one of claims 38-42, wherein the dosage form is a tablet having an average breaking force of more than 50 N.

44. The dosage form of any one of claims 38-43, wherein the dosage form has faster dissolution than a comparative dosage form that differs only by having been made from a physical mix of the darunavir and surfactant, as determined by the time required to release 80% of the darunavir from the dosage form, when tested using any one of the following:
(1) a USP II apparatus, 900 ml vessel, 75 RPM, using deionized water;
(2) a USP II apparatus, 900 ml vessel, 75 RPM, using pH 2 simulated gastric fluid; or
(3) a USP II apparatus, 900 ml vessel, 75 RPM, using pH 6.8 buffer.
